(19) United States
(12) Patent Application Publication
(10) Pub. No.: US 2013/0317073 A1
(43) Pub. Date: Nov. 28, 2013

(54) METHODS OF PEST CONTROL IN SOYBEAN

(71) Applicants: Christoph Vock, Basel (CH); Jerome Yves Cassayre, Stein (CH); Myriem El Qacemi, Stein (CH)

(72) Inventors: Christoph Vock, Basel (CH); Jerome Yves Cassayre, Stein (CH); Myriem El Qacemi, Stein (CH)

(73) Assignees: SYNGENTA LIMITED, Guildford, Surrey (GB); SYNGENTA PARTICIPATIONS AG, Basel (CH)

(21) Appl. No.: 13/983,491

(22) PCT Filed: Feb. 1, 2012
(86) PCT No.: PCT/EP12/51638

§ 371 Date: Aug. 2, 2013

(30) Foreign Application Priority Data
Feb. 3, 2011 (EP) 11158945.3
Feb. 3, 2011 (EP) PCT/EP2011/051511
Feb. 3, 2011 (EP) PCT/EP2011/051512
Feb. 3, 2011 (EP) PCT/EP2011/051513
Aug. 25, 2011 (EP) 111789459

(51) Int. Cl. A01N 43/80; C07D 261/04; C07D 413/12

(52) U.S. Cl. CPC A01N 43/80; C07D 413/12; C07D 261/04
USPC 514/378

(57) ABSTRACT

The present invention provides methods comprising applying to a crop of soybean plants, the locus thereof, or propagation material thereof, a compound a compound of formula III wherein A³, A⁴, A^{4′} and A^{5′} are independently C—H, or nitrogen and wherein #1 indicates the bond to X and #2 indicates the bond to cycle B; cycle B is selected from B1 to B6 wherein #1 indicates the bond to cycle A, #2 indicates the bond to R⁷ and #3 indicates the bond to cycle C; cycle C is phenyl; R⁵ is chloro, bromo, CF₃ or methyl; R⁷ is chlorodifluoromethyl or trifluoromethyl; each R⁸ is independently bromo, chloro, fluoro or trifluoromethyl; p is 2 or 3; and wherein X is defined in the claims. The methods are preferably for the control of stinkbugs, in particular Euschistus.

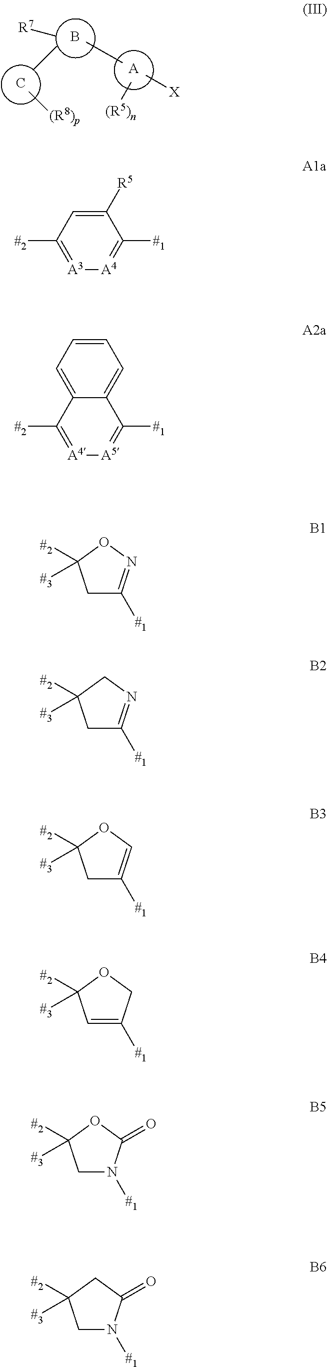

The present invention relates to methods of pest control in soybean crops.

Stink bugs (Hemiptera Pentatomidae) are true bugs which can be significant pests when present in large numbers. The nymphs and adults have piercing mouthparts which most use to suck sap from plants. According to Stewart et al., Soybean Insects—Stink bugs, University of Tennessee Institute of Agriculture, W200 09-0098, stink bugs are probably the most common pest problem in soybean. Although they may feed on many parts of the plant, they typically target developing seed including the pods, meaning that injury to soybean seed is the primary problem associated with stink bug infestations.

Of the complex of sucking bugs that occur in cultivation, the brown stinkbug Euschistus heros is currently considered to be the most abundant species in northern Parana to Central Brazil (Corrêa-Ferreira & Panizzi, 1999), and is a significant problem in soybean (Schmidt et al., 2003). The bugs occur in soybeans from the vegetative stage and are harmful from the beginning of pod formation until grain maturity. They cause damage to the seed (Galileo & Heinrichs 1978a, Panizzi & Slansky Jr., 1985) and can also open the way to fungal diseases and cause physiological disorders, such as soybean leaf retention (Galileo & Heinrichs 1978, Todd & Herzog, 1980).

Control of stinkbugs in soybean is often vital to prevent significant economic damage. Insecticides commonly used to control stinkbugs include pyrethroids, neonicotinoids and organophosphates, although pyrethroid insecticides are usually the method of choice for controlling stink bugs in soybean. However, there are increasing problems with insecticide resistance, particularly in brown stink bug populations and particularly to pyrethroids. Euschistus heros (F.)) can also be difficult to manage using organophosphates or endosulfan (Sosa-Gomez et al., 2009). There is therefore a need for effective alternative methods of controlling stinkbugs in soybean.

Compounds that are insecticidally, acaricidally, nematicidally and/or moluscicidally active by antagnonism of the gamma-aminobutyric acid (GABA)-gated chloride channel, and which comprise a partially saturated heterocycle that is substituted by a haloalkyl substituent and one or two optionally substituted aromatic or heteroaromatic rings, represent a new class of pesticides that are described for example in Ozoe et al. Biochemical and Biophysical Research Communications, 391 (2010) 744-749. Compounds from this class are broadly described in WO 2005/085216 (EP1731512), WO 2007/123853, WO 2007/075459, WO2009/002809, WO 2008/019760, WO 2008/122375, WO 2008/128711, WO 2009/097992, WO 2010/072781, WO 2010/072781, WO 2008/126665, WO 2007/125984, WO 2008/130651, JP 2008110971, JP2008133273, JP2009108046, WO2009/022746, WO 2009/022746, WO 2010/032437, WO2009/080250, WO2010/020521, WO2010/025998, WO2010/020522, WO2010/084067, WO2010/086225, WO2010/149506 and WO2010/108733.

It has now surprisingly been found that particular insecticides from this new class of gamma-aminobutyric acid (GABA)-gated chloride channel antagonists (disclosed in e.g. WO 2005/085216 (EP1731512), WO2009/002809 and WO2009/080250) are highly effective at controlling stinkbugs, and in some cases provide greater control than the current market standard. It has also surprisingly been found that these compounds exhibit significantly higher activity against stinkbugs than structurally similar compounds. These compounds therefore represent an important new solution for safeguarding soybean crops from stinkbugs, particularly where stink bugs are resistant to current methods.

In a first aspect the invention provides a method comprising applying to a crop of soybean plants, the locus thereof, or propagation material thereof, a compound a compound of formula I

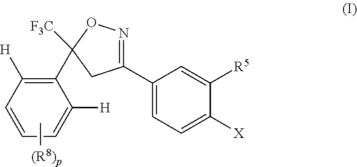

wherein X is P1 or P2

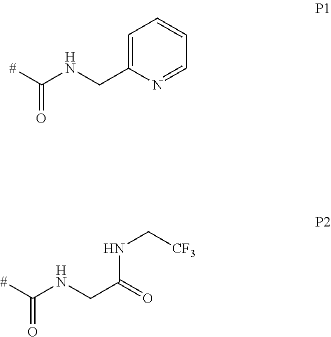

R⁵ is chloro, bromo, CF₃ or methyl;

each R⁸ is independently bromo, chloro, fluoro or trifluoromethyl;

p is 1, 2 or 3;

and wherein the method is for controlling and/or preventing infestation of the soybean crop by Euschistus, preferably Euschistus heros.
In a further aspect the invention provides use of a compound of formula I for control of Euschistus, preferably Euschistus heros. The use may be for controlling stinkbugs (Euschistus) that are resistant to one or more other insecticides, preferably pyrethroid, neonicotinoids and organophosphates, more preferably pyrethroid insecticides.

In a further aspect the invention provides a method comprising applying to a crop of soybean plants, the locus thereof, or propagation material thereof, a compound of formula II

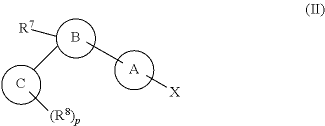

wherein

cycle A is A2a

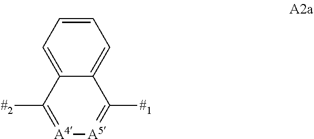

wherein A^{4′} and A^{5′} are independently C—H, or nitrogen and wherein #1 indicates the bond to X and #2 indicates the bond to cycle B;

cycle B is selected from B1 to B6

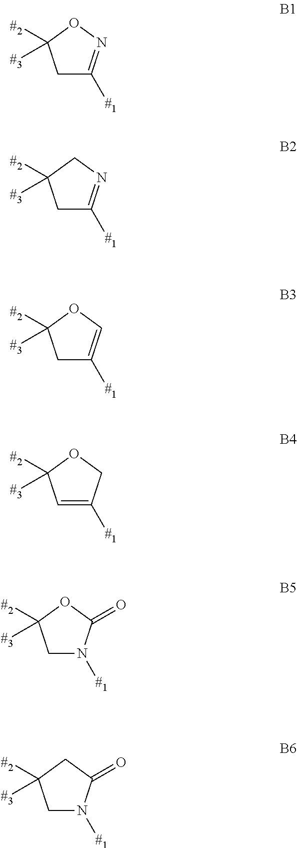

wherein #1 indicates the bond to cycle A, #2 indicates the bond to R⁷ and #3 indicates the bond to cycle C;

cycle C is phenyl;

R⁷ is chlorodifluoromethyl or trifluoromethyl;

each R⁸ is independently bromo, chloro, fluoro or trifluoromethyl;

p is 2 or 3; and

X is P1 or P2

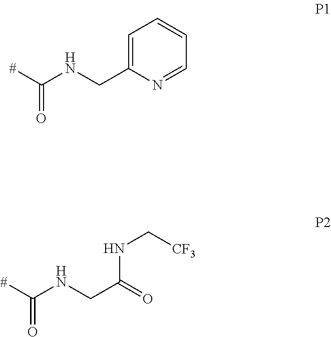

and wherein the method is for controlling and/or preventing infestation of the soybean crop by stinkbugs.

The stinkbugs may be stinkbugs that are resistant to one or more other insecticides, preferably pyrethroid, neonicotinoids and organophosphates, more preferably pyrethroid insecticides.

In a further aspect the invention provides a method comprising applying to a crop of soybean plants, the locus thereof, or propagation material thereof, compound of formula III

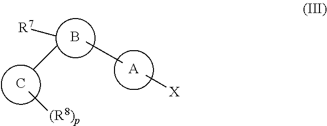

wherein

cycle A is A1a or A2a

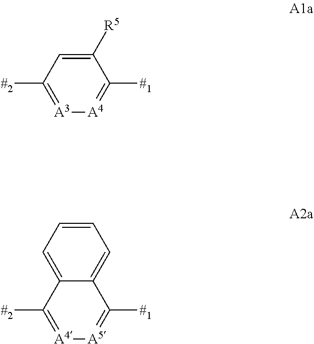

wherein A³, A⁴, A^{4′} and A^{5′} are independently C—H, or nitrogen and wherein #1 indicates the bond to X and #2 indicates the bond to cycle B;

cycle B is selected from B1 to B6

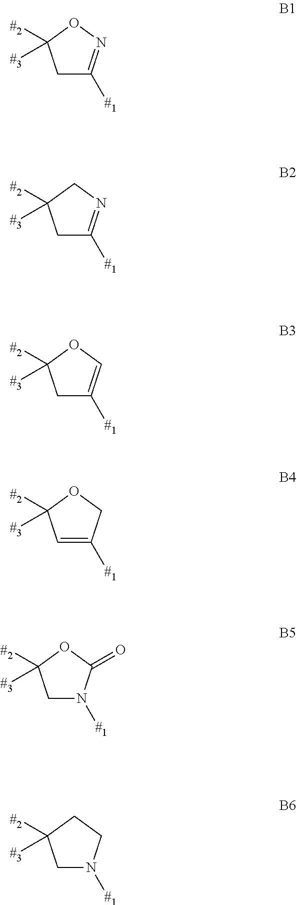

wherein #1 indicates the bond to cycle A, #2 indicates the bond to R⁷ and #3 indicates the bond to cycle C;

cycle C is phenyl;

R⁵ is chloro, bromo, CF₃ or methyl;

R⁷ is chlorodifluoromethyl or trifluoromethyl;

each R⁸ is independently bromo, chloro, fluoro or trifluoromethyl;

p is 2 or 3;

and X is selected from P3 to P11

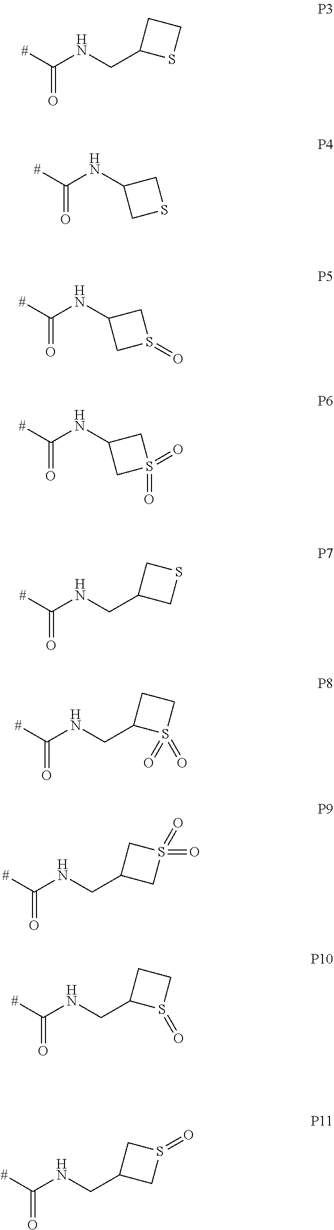

In a further aspect the invention provides a method of controlling and/or preventing infestation of stinkbugs in soybean comprising applying to a crop of soybean plants, the locus thereof, or propagation material thereof, a compound that of formula III. The stinkbugs may be stinkbugs that are resistant to one or more other insecticides, preferably pyrethroid, neonicotinoids and organophosphates, more preferably pyrethroid insecticides.

In a further aspect the invention provides use of a compound of formula III for control of stinkbugs. The use may be for controlling stinkbugs that are resistant to one or more other insecticides, preferably pyrethroid, neonicotinoids and organophosphates, more preferably pyrethroid insecticides.

The compounds of the invention may exist in different geometric or optical isomers or tautomeric forms. This invention covers all such isomers and tautomers and mixtures thereof in all proportions as well as isotopic forms such as deuterated compounds. The compounds of the invention may contain one or more asymmetric carbon atoms, for example, at the C(#2)#3 group, and may exist as enantiomers (or as pairs of diastereoisomers) or as mixtures of such.

In one group of compounds of formula I X is P1 or P2, R⁵ is chloro, bromo, CF₃ or methyl;

each R⁸ is independently bromo, chloro, fluoro or trifluoromethyl; p is 2 or 3. Preferably X is P1. Each R⁸ may take the same value.

A particularly preferred compound of formula I is a compound of formula Ix

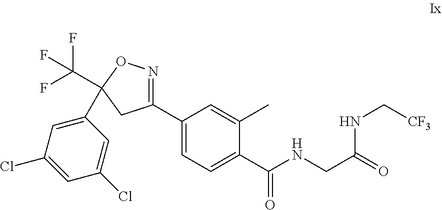 [SMILES: Cc1cc(C2=NOC(c3cc(Cl)cc(Cl)c3)(C(F)(F)F)C2)ccc1C(=O)NCC(=O)NCC(F)(F)F]

Another particularly preferred compound of formula I is a compound of formula Iy

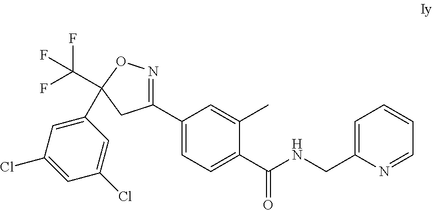 [SMILES: Cc1cc(C2=NOC(c3cc(Cl)cc(Cl)c3)(C(F)(F)F)C2)ccc1C(=O)NCc1ccccn1]

Preferred values of cycle A, cycle B, cycle C, X, p, A¹, A², A^{4′}, A^{5′}, R⁵, R⁷ and R⁸ in compounds of formula II are, in any combination, as set out below.

Preferably cycle B is a cycle selected from cycle B1, B2 and B3, more preferably B1.

In one group of compounds cycle B is B 1. In another group of compounds cycle B is B2, in another group of compounds cycle B is B3, in another group of compounds cycle B is B4, in another group of compounds cycle B is B5, in another group of compounds cycle B is B6.

Preferably cycle C is cycle C1

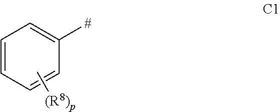

More preferably cycle C is 3,5-dibromo-phenyl, 3,5-dichloro-phenyl, 3,4-dichloro-phenyl, 3,5-dichloro-4-fluoro-phenyl or 3,4,5-trichloro-phenyl.

Preferably A³ is C—H or C—R⁵, most preferably A³ is C—H.

Preferably A⁴ is C—H or C—R⁵, most preferably A⁴ is C—H.

Preferably A^{4′} is C—H or C—R⁵, most preferably A^{4′} is C—H.

Preferably ^{5′} is C—H or C—R⁵, most preferably A^{4′} is C—H.

Preferably no more than one of A³ and A⁴ is nitrogen. Preferably no more than one of A^{4′} and A^{5′} is nitrogen.

Preferably R⁵ is methyl.

Preferably R⁷ is trifluoromethyl.

Preferably each R⁸ is independently bromo or chloro.

Preferably p is 2.

Preferably X is P4, P5 or P6.

Preferred values of cycle B, cycle C, X, p, A¹, A², A^{4′}, A^{5′}, R⁵, R⁷ and R⁸ in compounds of formula III are, in any combination, as set out for compounds of formula II.

In compounds of formula III preferably cycle A is cycle Ala in which A³ and A⁴ are C—H.

In one group of compounds of formula II each R⁸ takes the same value. Likewise, in one group of compounds of formula III each R⁸ takes the same value.

The following tables illustrate specific compounds of the invention:

### Table 1:

Table 1 provides 176 compounds of formula (A) wherein G is oxygen, R⁷ is trifluoromethyl, R⁵ is methyl, R¹ is hydrogen, and cycle C and R² have the values listed Table X below.

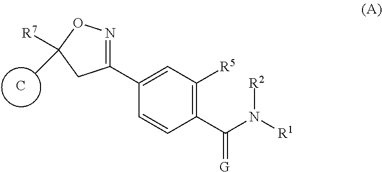

### Table 2:

Table 2 provides 176 compounds of formula (B) wherein G is oxygen, R⁷ is trifluoromethyl, R⁵ is methyl, R¹ is hydrogen, and cycle C and R² have the values listed Table X below.

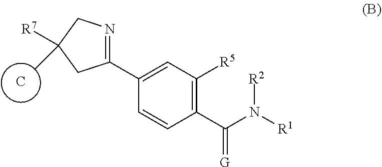

### Table 3:

Table 3 provides 176 compounds of formula (C) wherein G is oxygen, R⁷ is trifluoromethyl, R⁵ is methyl, R¹ is hydrogen, and cycle C and R² have the values listed Table X below.

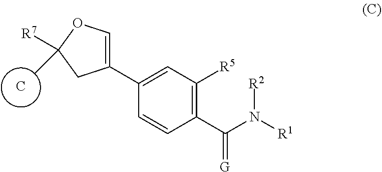

### Table 4:

Table 4 provides 176 compounds of formula (D) wherein G is oxygen, R⁷ is trifluoromethyl, R⁵ is methyl, R¹ is hydrogen, and cycle C and R² have the values listed Table X below.

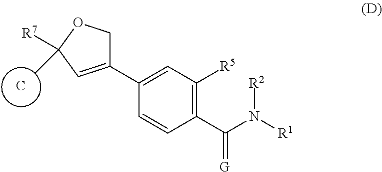

### Table 5:

Table 5 provides 176 compounds of formula (E) wherein G is oxygen, R⁷ is trifluoromethyl, R⁵ is methyl, R¹ is hydrogen, and cycle C and R² have the values listed Table X below.

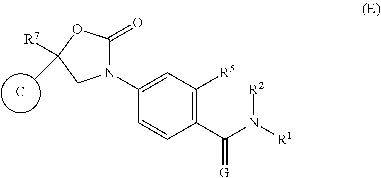

### Table 6:

Table 6 provides 176 compounds of formula (F) wherein G is oxygen, R⁷ is trifluoromethyl, R⁵ is methyl, R¹ is hydrogen, and cycle C and R² have the values listed Table X below.

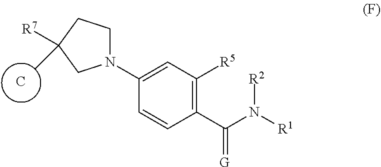

### Table 7

Table 7 provides 176 compounds of formula (G) wherein G is oxygen, R⁷ is trifluoromethyl, R¹ is hydrogen, and cycle C and R² have the values listed Table X below.

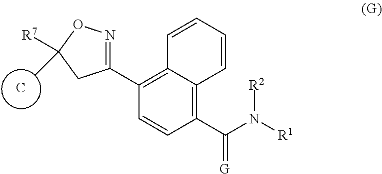

TABLE X

| | Cycle C | R² |
|---|---|---|
| X. 1 | 3,5-dichloro-phenyl- | (2,2,2-trifluoro-ethylcarbamoyl)-methyl |
| X. 2 | 3,5-dichloro-phenyl- | (pyrid-2-yl)-methyl- |
| X. 3 | 3,5-dichloro-phenyl- | thietan-3-yl- |
| X. 4 | 3,5-dichloro-phenyl- | 1-oxo-thietan-3-yl- |
| X. 5 | 3,5-dichloro-phenyl- | 1,1-dioxo-thietan-3-yl- |
| X. 6 | 3,5-dichloro-phenyl- | thietan-2-yl-methyl- |
| X. 7 | 3,5-dichloro-phenyl- | 1-oxo-thietan-2-yl-methyl- |
| X. 8 | 3,5-dichloro-phenyl- | 1,1-dioxo-thietan-2-yl-methyl- |
| X. 9 | 3,5-dichloro-phenyl- | thietan-3-yl-methyl- |
| X. 10 | 3,5-dichloro-phenyl- | 1-oxo-thietan-3-yl-methyl- |
| X. 11 | 3,5-dichloro-phenyl- | 1,1-dioxo-thietan-3-yl-methyl- |
| X. 12 | 3,4,5-trichloro-phenyl- | (2,2,2-trifluoro-ethylcarbamoyl)-methyl |
| X. 13 | 3,4,5-trichloro-phenyl- | (pyrid-2-yl)-methyl- |
| X. 14 | 3,4,5-trichloro-phenyl- | thietan-3-yl- |
| X. 15 | 3,4,5-trichloro-phenyl- | 1-oxo-thietan-3-yl- |
| X. 16 | 3,4,5-trichloro-phenyl- | 1,1-dioxo-thietan-3-yl- |
| X. 17 | 3,4,5-trichloro-phenyl- | thietan-2-yl-methyl- |
| X. 18 | 3,4,5-trichloro-phenyl- | 1-oxo-thietan-2-yl-methyl- |
| X. 19 | 3,4,5-trichloro-phenyl- | 1,1-dioxo-thietan-2-yl-methyl- |
| X. 20 | 3,4,5-trichloro-phenyl- | thietan-3-yl-methyl- |
| X. 21 | 3,4,5-trichloro-phenyl- | 1-oxo-thietan-3-yl-methyl- |
| X. 22 | 3,4,5-trichloro-phenyl- | 1,1-dioxo-thietan-3-yl-methyl- |
| X. 23 | 3,5-dichloro-4-fluoro-phenyl- | (2,2,2-trifluoro-ethylcarbamoyl)-methyl |
| X. 24 | 3,5-dichloro-4-fluoro-phenyl- | (pyrid-2-yl)-methyl- |
| X. 25 | 3,5-dichloro-4-fluoro-phenyl- | thietan-3-yl- |
| X. 26 | 3,5-dichloro-4-fluoro-phenyl- | 1-oxo-thietan-3-yl- |
| X. 27 | 3,5-dichloro-4-fluoro-phenyl- | 1,1-dioxo-thietan-3-yl- |
| X. 28 | 3,5-dichloro-4-fluoro-phenyl- | thietan-2-yl-methyl- |
| X. 29 | 3,5-dichloro-4-fluoro-phenyl- | 1-oxo-thietan-2-yl-methyl- |
| X. 30 | 3,5-dichloro-4-fluoro-phenyl- | 1,1-dioxo-thietan-2-yl-methyl- |
| X. 31 | 3,5-dichloro-4-fluoro-phenyl- | thietan-3-yl-methyl- |
| X. 32 | 3,5-dichloro-4-fluoro-phenyl- | 1-oxo-thietan-3-yl-methyl- |
| X. 33 | 3,5-dichloro-4-fluoro-phenyl- | 1,1-dioxo-thietan-3-yl-methyl- |
| X. 34 | 3-chloro-4-fluoro-phenyl- | (2,2,2-trifluoro-ethylcarbamoyl)-methyl |
| X. 35 | 3-chloro-4-fluoro-phenyl- | (pyrid-2-yl)-methyl- |
| X. 36 | 3-chloro-4-fluoro-phenyl- | thietan-3-yl- |
| X. 37 | 3-chloro-4-fluoro-phenyl- | 1-oxo-thietan-3-yl- |
| X. 38 | 3-chloro-4-fluoro-phenyl- | 1,1-dioxo-thietan-3-yl- |
| X. 39 | 3-chloro-4-fluoro-phenyl- | thietan-2-yl-methyl- |
| X. 40 | 3-chloro-4-fluoro-phenyl- | 1-oxo-thietan-2-yl-methyl- |
| X. 41 | 3-chloro-4-fluoro-phenyl- | 1,1-dioxo-thietan-2-yl-methyl- |
| X. 42 | 3-chloro-4-fluoro-phenyl- | thietan-3-yl-methyl- |
| X. 43 | 3-chloro-4-fluoro-phenyl- | 1-oxo-thietan-3-yl-methyl- |
| X. 44 | 3-chloro-4-fluoro-phenyl- | 1,1-dioxo-thietan-3-yl-methyl- |
| X. 45 | 3-chloro-4-fluoro-phenyl- | (2,2,2-trifluoro-ethylcarbamoyl)-methyl |
| X. 46 | 3-fluoro-4-chloro-phenyl- | (pyrid-2-yl)-methyl- |
| X. 47 | 3-fluoro-4-chloro-phenyl- | thietan-3-yl- |
| X. 48 | 3-fluoro-4-chloro-phenyl- | 1-oxo-thietan-3-yl- |
| X. 49 | 3-fluoro-4-chloro-phenyl- | 1,1-dioxo-thietan-3-yl- |
| X. 50 | 3-fluoro-4-chloro-phenyl- | thietan-2-yl-methyl- |
| X. 51 | 3-fluoro-4-chloro-phenyl- | 1-oxo-thietan-2-yl-methyl- |
| X. 52 | 3-fluoro-4-chloro-phenyl- | 1,1-dioxo-thietan-2-yl-methyl- |
| X. 53 | 3-fluoro-4-chloro-phenyl- | thietan-3-yl-methyl- |
| X. 54 | 3-fluoro-4-chloro-phenyl- | 1-oxo-thietan-3-yl-methyl- |
| X. 55 | 3-fluoro-4-chloro-phenyl- | 1,1-dioxo-thietan-3-yl-methyl- |
| X. 56 | 3,4-dichloro-phenyl- | (2,2,2-trifluoro-ethylcarbamoyl)-methyl |
| X. 57 | 3,4-dichloro-phenyl- | (pyrid-2-yl)-methyl- |
| X. 58 | 3,4-dichloro-phenyl- | thietan-3-yl- |
| X. 59 | 3,4-dichloro-phenyl- | 1-oxo-thietan-3-yl- |
| X. 60 | 3,4-dichloro-phenyl- | 1,1-dioxo-thietan-3-yl- |
| X. 61 | 3,4-dichloro-phenyl- | thietan-2-yl-methyl- |
| X. 62 | 3,4-dichloro-phenyl- | 1-oxo-thietan-2-yl-methyl- |
| X. 63 | 3,4-dichloro-phenyl- | 1,1-dioxo-thietan-2-yl-methyl- |
| X. 64 | 3,4-dichloro-phenyl- | thietan-3-yl-methyl- |
| X. 65 | 3,4-dichloro-phenyl- | 1-oxo-thietan-3-yl-methyl- |
| X. 66 | 3,4-dichloro-phenyl- | 1,1-dioxo-thietan-3-yl-methyl- |
| X. 67 | 3,5-dichloro-4-bromo-phenyl- | (2,2,2-trifluoro-ethylcarbamoyl)-methyl |
| X. 68 | 3,5-dichloro-4-bromo-phenyl- | (pyrid-2-yl)-methyl- |
| X. 69 | 3,5-dichloro-4-bromo-phenyl- | thietan-3-yl- |
| X. 70 | 3,5-dichloro-4-bromo-phenyl- | 1-oxo-thietan-3-yl- |
| X. 71 | 3,5-dichloro-4-bromo-phenyl- | 1,1-dioxo-thietan-3-yl- |
| X. 72 | 3,5-dichloro-4-bromo-phenyl- | thietan-2-yl-methyl- |
| X. 73 | 3,5-dichloro-4-bromo-phenyl- | 1-oxo-thietan-2-yl-methyl- |
| X. 74 | 3,5-dichloro-4-bromo-phenyl- | 1,1-dioxo-thietan-2-yl-methyl- |
| X. 75 | 3,5-dichloro-4-bromo-phenyl- | thietan-3-yl-methyl- |
| X. 76 | 3,5-dichloro-4-bromo-phenyl- | 1-oxo-thietan-3-yl-methyl- |
| X. 77 | 3,5-dichloro-4-bromo-phenyl- | 1,1-dioxo-thietan-3-yl-methyl- |
| X. 78 | 3,5-dichloro-4-fluoro-phenyl- | (2,2,2-trifluoro-ethylcarbamoyl)-methyl |
| X. 79 | 3,5-dichloro-4-fluoro-phenyl- | (pyrid-2-yl)-methyl- |
| X. 80 | 3,5-dichloro-4-fluoro-phenyl- | thietan-3-yl- |
| X. 81 | 3,5-dichloro-4-fluoro-phenyl- | 1-oxo-thietan-3-yl- |
| X. 82 | 3,5-dichloro-4-fluoro-phenyl- | 1,1-dioxo-thietan-3-yl- |
| X. 83 | 3,5-dichloro-4-fluoro-phenyl- | thietan-2-yl-methyl- |
| X. 84 | 3,5-dichloro-4-fluoro-phenyl- | 1-oxo-thietan-2-yl-methyl- |
| X. 85 | 3,5-dichloro-4-fluoro-phenyl- | 1,1-dioxo-thietan-2-yl-methyl- |
| X. 86 | 3,5-dichloro-4-fluoro-phenyl- | thietan-3-yl-methyl- |
| X. 87 | 3,5-dichloro-4-fluoro-phenyl- | 1-oxo-thietan-3-yl-methyl- |
| X. 88 | 3,5-dichloro-4-fluoro-phenyl- | 1,1-dioxo-thietan-3-yl-methyl- |
| X. 89 | 3,4,5-trifluoro-phenyl- | (2,2,2-trifluoro-ethylcarbamoyl)-methyl |
| X. 90 | 3,4,5-trifluoro-phenyl- | (pyrid-2-yl)-methyl- |
| X. 91 | 3,4,5-trifluoro-phenyl- | thietan-3-yl- |
| X. 92 | 3,4,5-trifluoro-phenyl- | 1-oxo-thietan-3-yl- |
| X. 93 | 3,4,5-trifluoro-phenyl- | 1,1-dioxo-thietan-3-yl- |
| X. 94 | 3,4,5-trifluoro-phenyl- | thietan-2-yl-methyl- |
| X. 95 | 3,4,5-trifluoro-phenyl- | 1-oxo-thietan-2-yl-methyl- |
| X. 96 | 3,4,5-trifluoro-phenyl- | 1,1-dioxo-thietan-2-yl-methyl- |
| X. 97 | 3,4,5-trifluoro-phenyl- | thietan-3-yl-methyl- |
| X. 98 | 3,4,5-trifluoro-phenyl- | 1-oxo-thietan-3-yl-methyl- |
| X. 99 | 3,4,5-trifluoro-phenyl- | 1,1-dioxo-thietan-3-yl-methyl- |
| X. 100 | 3-chloro-5-bromo-phenyl- | (2,2,2-trifluoro-ethylcarbamoyl)-methyl |
| X. 101 | 3-chloro-5-bromo-phenyl- | (pyrid-2-yl)-methyl- |
| X. 102 | 3-chloro-5-bromo-phenyl- | thietan-3-yl- |
| X. 103 | 3-chloro-5-bromo-phenyl- | 1-oxo-thietan-3-yl- |
| X. 104 | 3-chloro-5-bromo-phenyl- | 1,1-dioxo-thietan-3-yl- |
| X. 105 | 3-chloro-5-bromo-phenyl- | thietan-2-yl-methyl- |
| X. 106 | 3-chloro-5-bromo-phenyl- | 1-oxo-thietan-2-yl-methyl- |
| X. 107 | 3-chloro-5-bromo-phenyl- | 1,1-dioxo-thietan-2-yl-methyl- |
| X. 108 | 3-chloro-5-bromo-phenyl- | thietan-3-yl-methyl- |
| X. 109 | 3-chloro-5-bromo-phenyl- | 1-oxo-thietan-3-yl-methyl- |
| X. 110 | 3-chloro-5-bromo-phenyl- | 1,1-dioxo-thietan-3-yl-methyl- |
| X. 111 | 3-chloro-5-fluoro-phenyl- | (2,2,2-trifluoro-ethylcarbamoyl)-methyl |
| X. 112 | 3-chloro-5-fluoro-phenyl- | (pyrid-2-yl)-methyl- |
| X. 113 | 3-chloro-5-fluoro-phenyl- | thietan-3-yl- |
| X. 114 | 3-chloro-5-fluoro-phenyl- | 1-oxo-thietan-3-yl- |
| X. 115 | 3-chloro-5-fluoro-phenyl- | 1,1-dioxo-thietan-3-yl- |
| X. 116 | 3-chloro-5-fluoro-phenyl- | thietan-2-yl-methyl- |
| X. 117 | 3-chloro-5-fluoro-phenyl- | 1-oxo-thietan-2-yl-methyl- |
| X. 118 | 3-chloro-5-fluoro-phenyl- | 1,1-dioxo-thietan-2-yl-methyl- |
| X. 119 | 3-chloro-5-fluoro-phenyl- | thietan-3-yl-methyl- |
| X. 120 | 3-chloro-5-fluoro-phenyl- | 1-oxo-thietan-3-yl-methyl- |
| X. 121 | 3-chloro-5-fluoro-phenyl- | 1,1-dioxo-thietan-3-yl-methyl- |
| X. 122 | 3-chloro-5-trifluoromethyl-phenyl- | (2,2,2-trifluoro-ethylcarbamoyl)-methyl |
| X. 123 | 3-chloro-5-trifluoromethyl-phenyl- | (pyrid-2-yl)-methyl- |
| X. 124 | 3-chloro-5-trifluoromethyl-phenyl- | thietan-3-yl- |
| X. 125 | 3-chloro-5-trifluoromethyl-phenyl- | 1-oxo-thietan-3-yl- |
| X. 126 | 3-chloro-5-trifluoromethyl-phenyl- | 1,1-dioxo-thietan-3-yl- |
| X. 127 | 3-chloro-5-trifluoromethyl-phenyl- | thietan-2-yl-methyl- |
| X. 128 | 3-chloro-5-trifluoromethyl-phenyl- | 1-oxo-thietan-2-yl-methyl- |
| X. 129 | 3-chloro-5-trifluoromethyl-phenyl- | 1,1-dioxo-thietan-2-yl-methyl- |
| X. 130 | 3-chloro-5-trifluoromethyl-phenyl- | thietan-3-yl-methyl- |
| X. 131 | 3-chloro-5-trifluoromethyl-phenyl- | 1-oxo-thietan-3-yl-methyl- |
| X. 132 | 3-chloro-5-trifluoromethyl-phenyl- | 1,1-dioxo-thietan-3-yl-methyl- |
| X. 133 | 3-chloro-4-chloro-5-trifluoromethyl-phenyl- | (2,2,2-trifluoro-ethylcarbamoyl)-methyl |
| X. 134 | 3-chloro-4-chloro-5-trifluoromethyl-phenyl- | (pyrid-2-yl)-methyl- |
| X. 135 | 3-chloro-4-chloro-5-trifluoromethyl-phenyl- | thietan-3-yl- |
| X. 136 | 3-chloro-4-chloro-5-trifluoromethyl-phenyl- | 1-oxo-thietan-3-yl- |
| X. 137 | 3-chloro-4-chloro-5-trifluoromethyl-phenyl- | 1,1-dioxo-thietan-3-yl- |
| X. 138 | 3-chloro-4-chloro-5-trifluoromethyl-phenyl- | thietan-2-yl-methyl- |
| X. 139 | 3-chloro-4-chloro-5-trifluoromethyl-phenyl- | 1-oxo-thietan-2-yl-methyl- |
| X. 140 | 3-chloro-4-chloro-5-trifluoromethyl-phenyl- | 1,1-dioxo-thietan-2-yl-methyl- |
| X. 141 | 3-chloro-4-chloro-5-trifluoromethyl-phenyl- | thietan-3-yl-methyl- |
| X. 142 | 3-chloro-4-chloro-5-trifluoromethyl-phenyl- | 1-oxo-thietan-3-yl-methyl- |
| X. 143 | 3-chloro-4-chloro-5-trifluoromethyl-phenyl- | 1,1-dioxo-thietan-3-yl-methyl- |
| X. 144 | 3,5-di-trifluoromethyl-phenyl- | (2,2,2-trifluoro-ethylcarbamoyl)-methyl |
| X. 145 | 3,5-di-trifluoromethyl-phenyl- | (pyrid-2-yl)-methyl- |
| X. 146 | 3,5-di-trifluoromethyl-phenyl- | thietan-3-yl- |
| X. 147 | 3,5-di-trifluoromethyl-phenyl- | 1-oxo-thietan-3-yl- |
| X. 148 | 3,5-di-trifluoromethyl-phenyl- | 1,1-dioxo-thietan-3-yl- |
| X. 149 | 3,5-di-trifluoromethyl-phenyl- | thietan-2-yl-methyl- |
| X. 150 | 3,5-di-trifluoromethyl-phenyl- | 1-oxo-thietan-2-yl-methyl- |
| X. 151 | 3,5-di-trifluoromethyl-phenyl- | 1,1-dioxo-thietan-2-yl-methyl- |
| X. 152 | 3,5-di-trifluoromethyl-phenyl- | thietan-3-yl-methyl- |
| X. 153 | 3,5-di-trifluoromethyl-phenyl- | 1-oxo-thietan-3-yl-methyl- |
| X. 154 | 3,5-di-trifluoromethyl-phenyl- | 1,1-dioxo-thietan-3-yl-methyl- |
| X. 155 | 3,5-di-trifluoromethyl-4-chloro-phenyl- | (2,2,2-trifluoro-ethylcarbamoyl)-methyl |
| X. 156 | 3,5-di-trifluoromethyl-4-chloro-phenyl- | (pyrid-2-yl)-methyl- |
| X. 157 | 3,5-di-trifluoromethyl-4-chloro-phenyl- | thietan-3-yl- |
| X. 158 | 3,5-di-trifluoromethyl-4-chloro-phenyl- | 1-oxo-thietan-3-yl- |
| X. 159 | 3,5-di-trifluoromethyl-4-chloro-phenyl- | 1,1-dioxo-thietan-3-yl- |
| X. 160 | 3,5-di-trifluoromethyl-4-chloro-phenyl- | thietan-2-yl-methyl- |
| X. 161 | 3,5-di-trifluoromethyl-4-chloro-phenyl- | 1-oxo-thietan-2-yl-methyl- |
| X. 162 | 3,5-di-trifluoromethyl-4-chloro-phenyl- | 1,1-dioxo-thietan-2-yl-methyl- |
| X. 163 | 3,5-di-trifluoromethyl-4-chloro-phenyl- | thietan-3-yl-methyl- |
| X. 164 | 3,5-di-trifluoromethyl-4-chloro-phenyl- | 1-oxo-thietan-3-yl-methyl- |
| X. 165 | 3,5-di-trifluoromethyl-4-chloro-phenyl- | 1,1-dioxo-thietan-3-yl-methyl- |
| X. 166 | 3-trifluoromethyl-phenyl- | (2,2,2-trifluoro-ethylcarbamoyl)-methyl |
| X. 167 | 3-trifluoromethyl-phenyl- | (pyrid-2-yl)-methyl- |
| X. 168 | 3-trifluoromethyl-phenyl- | thietan-3-yl- |
| X. 169 | 3-trifluoromethyl-phenyl- | 1-oxo-thietan-3-yl- |
| X. 170 | 3-trifluoromethyl-phenyl- | 1,1-dioxo-thietan-3-yl- |
| X. 171 | 3-trifluoromethyl-phenyl- | thietan-2-yl-methyl- |
| X. 172 | 3-trifluoromethyl-phenyl- | 1-oxo-thietan-2-yl-methyl- |
| X. 173 | 3-trifluoromethyl-phenyl- | 1,1-dioxo-thietan-2-yl-methyl- |
| X. 174 | 3-trifluoromethyl-phenyl- | thietan-3-yl-methyl- |
| X. 175 | 3-trifluoromethyl-phenyl- | 1-oxo-thietan-3-yl-methyl- |
| X. 176 | 3-trifluoromethyl-phenyl- | 1,1-dioxo-thietan-3-yl-methyl- |

Compounds of formula I include at least one chiral centre and may exist as compounds of formula I* or compounds of formula I**.

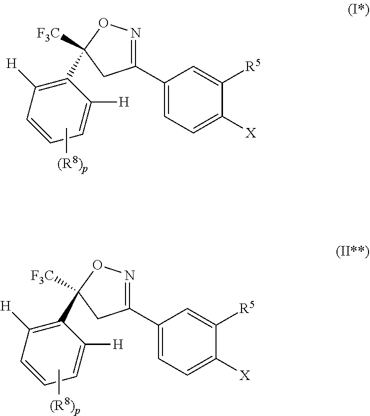

Compounds of formula II where cycle B is selected from B1 to B6 include at least one chiral centre and may exist as compounds of formula II* (IIA*, IIB*, IIC*, IID*, IIE*, IIF*) or compounds of formula II** (IIA**, IIB**, IIC**, IID**, IIE**, IIF**).

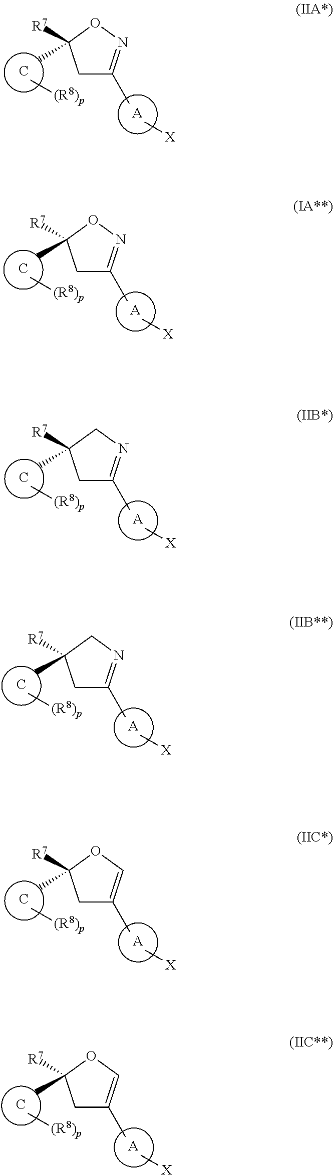

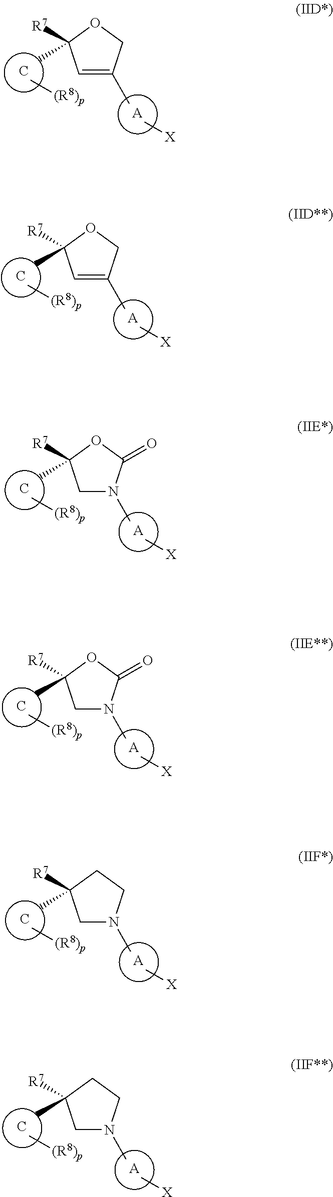

Compounds of formula III where cycle B is selected from B1 to B6 include at least one chiral centre and may exist as compounds of formula III* (IIIA*, IIIB*, IIIC*, IIID*, IIIE*, IIIF*) or compounds of formula III** (IIIA**, IIIB**, IIIC**, IIID**, IIIE**, IIIF**). These generic structures correspond to the structures of formula II above.

Generally the compounds of formula I** are more biologically active than the respective compounds of formula I*. The invention includes mixtures of compounds I* and I** in any ratio e.g. in a molar ratio of 1:99 to 99:1, e.g. 10:1 to 1:10, e.g. a substantially 50:50 molar ratio. In an enantiomerically (or epimerically) enriched mixture of formula I**, the molar proportion of compound I** compared to the total amount of both enantiomers is for example greater than 50%, e.g. at least 55, 60, 65, 70, 75, 80, 85, 90, 95, 96, 97, 98, or at least 99%. Likewise, in enantiomerically (or epimerically) enriched mixture of formula II*, the molar proportion of the compound of formula II* compared to the total amount of both enantiomers (or epimerically) is for example greater than 50%, e.g. at least 55, 60, 65, 70, 75, 80, 85, 90, 95, 96, 97, 98, or at least 99%. Enantiomerically (or epimerically) enriched mixtures of formula I** are preferred.

Generally the compounds of formula II** are more biologically active than the respective compounds of formula II*. The invention includes mixtures of compounds II* and II** in any ratio e.g. in a molar ratio of 1:99 to 99:1, e.g. 10:1 to 1:10, e.g. a substantially 50:50 molar ratio. In an enantiomerically (or epimerically) enriched mixture of formula II**, the molar proportion of compound II** compared to the total amount of both enantiomers is for example greater than 50%, e.g. at least 55, 60, 65, 70, 75, 80, 85, 90, 95, 96, 97, 98, or at least 99%. Likewise, in enantiomerically (or epimerically) enriched mixture of formula II*, the molar proportion of the compound of formula II* compared to the total amount of both enantiomers (or epimerically) is for example greater than 50%, e.g. at least 55, 60, 65, 70, 75, 80, 85, 90, 95, 96, 97, 98, or at least 99%. Enantiomerically (or epimerically) enriched mixtures of formula II** are preferred.

Generally the compounds of formula III** are more biologically active than the respective compounds of formula III*. The invention includes mixtures of compounds III* and III** in any ratio e.g. in a molar ratio of 1:99 to 99:1, e.g. 10:1 to 1:10, e.g. a substantially 50:50 molar ratio. In an enantiomerically (or epimerically) enriched mixture of formula III**, the molar proportion of compound III** compared to the total amount of both enantiomers is for example greater than 50%, e.g. at least 55, 60, 65, 70, 75, 80, 85, 90, 95, 96, 97, 98, or at least 99%. Likewise, in enantiomerically (or epimerically) enriched mixture of formula III*, the molar proportion of the compound of formula III* compared to the total amount of both enantiomers (or epimerically) is for example greater than 50%, e.g. at least 55, 60, 65, 70, 75, 80, 85, 90, 95, 96, 97, 98, or at least 99%. Enantiomerically (or epimerically) enriched mixtures of formula III** are preferred.

A preferred compound of the invention is a compound of formula Ix**

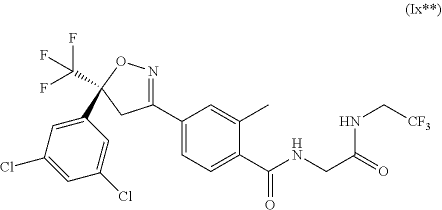 [SMILES: Cc1cc(C2=NO[C@@](c3cc(Cl)cc(Cl)c3)(C(F)(F)F)C2)ccc1C(=O)NCC(=O)NCC(F)(F)F]

The compounds of the invention can be made according to the methods described in the patent applications listed above. Additional methods can be found in PCT/EP2011/051284.

In a further aspect the invention also provides a method comprising applying to a crop of soybean plants, the locus thereof, or propagation material thereof, a compound of formula IV

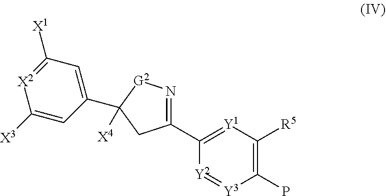

wherein P is P0

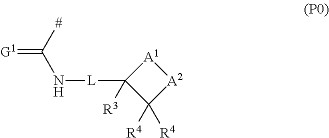

G¹ is oxygen;

### G² is O or CH₂;

L is a bond, methylene or ethylene;
one of A¹ and A² is S, SO or SO₂ and the other is —C(R⁴)R⁴—;
R³ is hydrogen;
each R⁴ is independently hydrogen or methyl;
Y¹ is C—R⁶, CH or nitrogen;
Y² and Y³ are independently CH or nitrogen;
wherein no more than two of Y¹, Y² and Y³ are nitrogen and wherein Y² and Y³ are not both nitrogen;
R⁵ is hydrogen, halogen, cyano, nitro, NH₂, C₁-C₂alkyl, C₁-C₂haloalkyl, C₃-C₅cycloalkyl, C₃-C₅halocycloalkyl, C₁-C₂alkoxy, C₁-C₂haloalkox_{Y};
R⁶ together with R⁵ forms a —CH═CH—CH═CH— bridge;
X² is C—X⁶ or nitrogen;
X¹, X³ and X⁶ are independently hydrogen, halogen or trihalomethyl, wherein at least two of X¹, X³ and X⁶ are not hydrogen;
X⁴ is trifluoromethyl, difluoromethyl or chlorodifluoromethyl.

In a further aspect the invention provides a method of controlling and/or preventing infestation of stinkbugs in soybean comprising applying to a crop of soybean plants, the locus thereof, or propagation material thereof, a compound of formula IV. Preferably the stinkbug is Euschistus, more preferably Euschistus heros. The stinkbugs may be stinkbugs that are resistant to one or more other insecticides, preferably pyrethroid, neonicotinoids and organophosphates, more preferably pyrethroid insecticides.

In a further aspect the invention provides use of a compound of formula IV for control of stinkbugs e.g. in soybean crops. Preferably the stinkbug is Euschistus, more preferably Euschistus heros. The use may be for controlling stinkbugs that are resistant to one or more other insecticides, preferably pyrethroid, neonicotinoids and organophosphates, more preferably pyrethroid insecticides.

Preferred values of P, G¹, G², A¹, A², R³, R⁴, X¹, X², X³ and X⁴ in compounds of formula IV are, in any combination, as set out below.

Preferably P is selected from P3 to P11

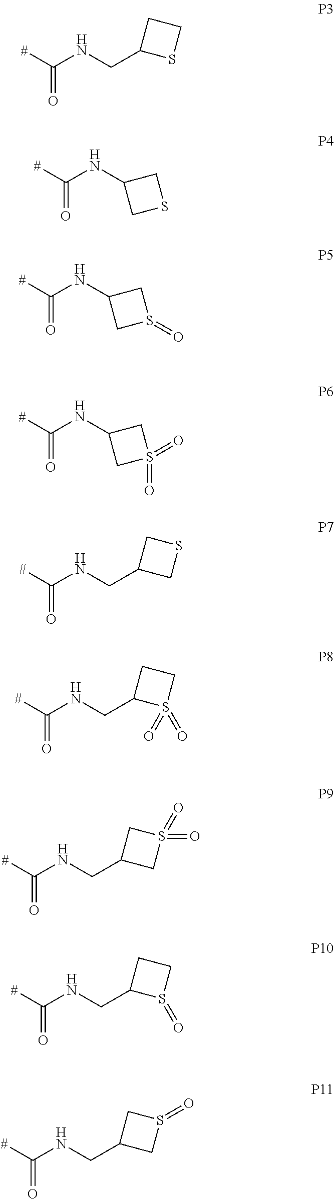

In one group of compounds P is selected from P4, P5 and P6.

Preferably each R⁴ is hydrogen.

Preferably R⁵ is hydrogen, chloro, bromo, fluoro, trifluoromethyl, methyl, ethyl, methoxy, nitro, trifluoromethoxy, cyano, cyclopropyl, more preferably R⁵ is hydrogen, chloro, bromo, fluoro, trifluoromethyl, methyl, ethyl, nitro, cyano, cyclopropyl, even more preferably R⁵ is chloro, bromo, fluoro, methyl, trifluoromethyl, most preferably methyl.

Preferably G¹ is oxygen.

Preferably G² is oxygen.

Preferably L is a bond.

Preferably A¹ is —C(R⁴)R⁴—, more preferably —CH₂—.

Preferably A² is S, SO or SO₂.

Preferably Y¹ is CH, Y² is CH, Y³ is CH, or Y¹ is N, Y² is CH, Y³ is CH, or Y¹ is N, Y² is N, Y³ is CH, or Y¹ is CH, Y² is N, Y³ is CH, or Y¹ is CH, Y² is CH, Y³ is N. Preferably Y¹ is CH, Y² is CH, and Y³ is CH.

Preferably X¹, X³ and X⁶ are independently hydrogen, halogen or trifluoromethyl, wherein at least two of X¹, X³ and X⁶ are not hydrogen. More preferably X¹, X³ and X⁶ are independently hydrogen, chloro, bromo or trifluoromethyl, wherein at least two of X¹, X³ and X⁶ are not hydrogen.

Preferably at least two of X¹, X³ and X⁶ are chloro, bromo or trifluoromethyl.

Preferably X² is C—X⁶;

Preferably X¹ is chloro, X² is CH, X³ is chloro, or X¹ is chloro, X² is C—F, X³ is hydrogen, or X¹ is fluoro, X² is C—Cl, X³ is hydrogen, or X¹ is chloro, X² is C—Cl, X³ is hydrogen, or X¹ is chloro, X^{2 is C—Br, X}³ is chloro, or X¹ is chloro, X² is C—F, X³ is chloro, or X¹ is chloro, X² is C—C¹, X³ is chloro, or X¹ is chloro, X² is C-I, X³ is chloro, or X¹ is fluoro, X² is C—F, X³ is fluoro, or X¹ is chloro, X² is CH, X³ is bromo, or X¹ is chloro, X² is CH, X³ is fluoro, or X¹ is chloro, X² is CH, X³ is trifluoromethyl, or X¹ is chloro, X² is C—C¹, X³ is trifluoromethyl, or X¹ is trifluoromethyl, X² is CH, X³ is trifluoromethyl, or X¹ is trifluoromethyl, X² is C—C¹, X³ is trifluoromethyl, or X¹ is trifluoromethyl, X² is CH, X³ is hydrogen, or X¹ is chloro, X² is N, X³ is chloro, or X¹ is trifluoromethyl, X² is N, X³ is trifluoromethyl. Most preferably X¹ is chloro, X² is CH, X³ is chloro.

Preferably X⁴ is trifluoromethyl, or chlorodifluoromethyl, more preferably trifluoromethyl.

In one group of compounds of formula IV G² is oxygen.

In one group of compounds of formula IV G² is CH₂.

In one group of compounds of formula IV Y¹ is C—R⁶ and R⁶ together with R⁵ forms a —CH═CH—CH═CH— bridge.

In one group of compounds of formula IV X² is C—X⁶, Y¹, Y² and Y³ are C—H, and R⁵ is chloro, bromo, methyl or trifluoromethyl.

In one group of compounds of formula IV X² is C—X⁶, Y¹, Y² and Y³ are C—H, G¹ is oxygen, G² is oxygen, A¹ is CH₂, A² is S, SO or SO₂, L is a bond, R³ and each R⁴ is hydrogen, R⁵ is chloro, bromo, methyl or trifluoromethyl.

In another group of compounds of formula IV X² is C—X⁶, Y¹, Y² and, are C—H, G¹ is oxygen, G² is oxygen, A¹ is CH₂, A² is S, SO or SO₂, L is a bond, R³ and each R⁴ is hydrogen, R⁵ is chloro, bromo, methyl or trifluoro methyl, Y¹ is CH, Y² is CH, and Y³ is CH, X⁴ is trifluoromethyl.

In another group of compounds of formula IV X² is C—X⁶, Y¹, Y² and Y³ are C—H, G¹ is oxygen, G² is oxygen, A¹ is CH₂, A² is S, SO or SO₂, L is a bond, R³ and each R⁴ is hydrogen, R⁵ is chloro, bromo, methyl or trifluoro methyl, Y¹ is CH, Y² is CH, and Y³ is CH, X⁴ is trifluoromethyl, X¹ is chloro, X² is CH, X³ is chloro.

In one group of compounds one of formula IV A¹ and A² is S, SO or SO₂ and the other is CH₂, L is a direct bond or methylene, Y² and Y³ are C—H or one of Y² and Y³ is C—H and the other is N; R³ is hydrogen or methyl; X¹ is Br, X² is CH and X³ is Br, or X¹ is C¹, X² is C—H and X³ is Cl, or X¹ is Cl, X² is C—C¹ and X³ is C—H, or X¹ is C¹, X² is C—C¹ and X³ is Cl; X⁴ is chlorodifluoromethyl or trifluoromethyl; R⁵ is methyl, Y¹ is C—R⁶, R⁶ is hydrogen, or R⁵ and R⁶ together form a bridging 1,3-butadiene group; each R⁴ is hydrogen; G¹ is oxygen; G² is oxygen.

Compounds of formula IV include at least one chiral centre and may exist as compounds of formula IV* or compounds of formula IV**.

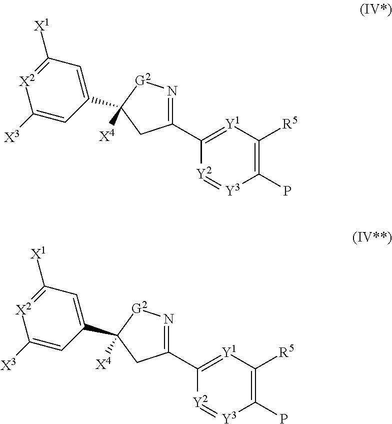

Compounds of formula IV** are more biologically active than compounds of formula IV*. The compound of formula IV may be a mixture of compounds IV* and IV** in any ratio e.g. in a molar ratio of 1:99 to 99:1, e.g. 10:1 to 1:10, e.g. a substantially 50:50 molar ratio. Preferably the compound of formula I is a racemic mixture of the compounds of formula IV** and IV* or is enantiomerically enriched for the compound of formula IV**. For example, when the compound of formula IV is an enantiomerically enriched mixture of formula IV**, the molar proportion of compound IV** compared to the total amount of both enantiomers is for example greater than 50%, e.g. at least 55, 60, 65, 70, 75, 80, 85, 90, 95, 96, 97, 98, or at least 99%. Preferably the compound of formula I is at least 90% enriched for the compound of formula I.

Where A¹ or A² is SO, the compound of formula IV may be a mixture of the cis and trans isomer in any ratio, e.g. in a molar ratio of 1:99 to 99:1, e.g. 10:1 to 1:10, e.g. a substantially 50:50 molar ratio. For example, in trans enriched mixtures of the compound of formula IV, e.g. when A¹ or A² is SO, the molar proportion of the trans compound in the mixture compared to the total amount of both cis and trans is for example greater than 50%, e.g. at least 55, 60, 65, 70, 75, 80, 85, 90, 95, 96, 97, 98, or at least 99%. Likewise, in cis enriched mixtures of the compound of formula IV (preferred), e.g. when A¹ or A² is SO, the molar proportion of the cis compound in the mixture compared to the total amount of both cis and trans is for example greater than 50%, e.g. at least 55, 60, 65, 70, 75, 80, 85, 90, 95, 96, 97, 98, or at least 99%. The compound of formula IV may be enriched for the trans sulphoxide. Likewise, the compound of formula IV may be enriched for the cis sulphoxide.

A selection of preferred compounds of formula IV are compounds A1 to A16 shown in Table A below.

TABLE A
Compounds of formula IV(a)
(IVa)

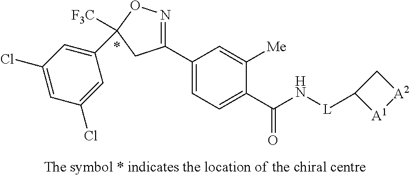

| Comp | Stereochemistry | | | |
|---|---|---|---|---|
| No. | at * | L | A² | A¹ |
| 1 | racemic mixture | bond | S | CH₂ |
| 2 | racemic mixture | bond | SO (cis) | CH₂ |
| 3 | racemic mixture | bond | SO (trans) | CH₂ |
| 4 | racemic mixture | bond | SO₂ | CH₂ |
| 5 | racemic mixture | CH₂ | CH₂ | S |
| 6 | racemic mixture | CH₂ | CH₂ | SO (cis) |
| 7 | racemic mixture | CH₂ | CH₂ | SO (trans) |
| 8 | racemic mixture | CH₂ | CH₂ | SO₂ |
| 9 | as for IV** | bond | S | CH₂ |
| 10 | as for IV** | bond | SO (cis) | CH₂ |
| 11 | as for IV** | bond | SO (trans) | CH₂ |
| 12 | as for IV** | bond | SO₂ | CH₂ |
| 13 | as for IV** | CH₂ | CH₂ | S |
| 14 | as for IV** | CH₂ | CH₂ | SO (cis) |
| 15 | as for IV** | CH₂ | CH₂ | SO (trans) |
| 16 | as for IV** | CH₂ | CH₂ | SO₂ |

IV** refers to a compound of formula IV**.

Reference to compounds of the invention also includes reference to salts and N-oxides.

The methods and uses of the invention are preferably for controlling and/or preventing infestation of the soybean crop by stink bugs, including stink bugs that are resistant to other insecticides, e.g. pyrethroid insecticides. Stinkbugs that are “resistant” to a particular insecticide refers e.g. to strains of stinkbugs that are less sensitive to that insecticide compared to the expected sensitivity of the same species of stinkbug. The expected sensitivity can be measured using e.g. a strain that has not previously been exposed to the insecticide.

Application is of the compounds of the invention is to a crop of soybean plants, the locus thereof or propagation material thereof. Preferably application is to a crop of soybean plants or the locus thereof, more preferably to a crop of soybean plants. Application may be before infestation or when the pest is present. Application of the compounds of the invention can be performed according to any of the usual modes of application, e.g. foliar, drench, soil, in furrow etc. However, control of stinkbugs is usually achieved by foliar application, which is the preferred mode of application according to the invention.

The compounds of the invention may be applied in combination with an attractant. An attractant is a chemical that causes the insect to migrate towards the location of application. For control of stinkbugs it can be advantageous to apply the compounds of the invention with an attractant, particularly when the application is foliar. Stinkbugs are often located near to the ground, and application of an attractant may encourage migration up the plant towards the active ingredient. Suitable attractants include glucose, sacchrose, salt, glutamate (e.g. Aji-no-moto™), citric acid (e.g. Orobor™), soybean oil, peanut oil and soybean milk. Glutamate and citric acid are of particular interest, with citric acid being preferred.

An attractant may be premixed with the compound of the invention prior to application, e.g. as a readymix or tankmix, or by simultaneous application or sequential application to the plant. Suitable rates of attractants are for example 0.02 kg/ha-3 kg/ha.

The compounds of the invention are preferably used for pest control on soybean at 1:500 g/ha, preferably 10-70 g/ha.

The compounds of the invention are suitable for use on any soybean plant, including those that have been genetically modified to be resistant to active ingredients such as herbicides, or to produce biologically active compounds that control infestation by plant pests.

The compounds of the invention are preferably used for pest control on soybean at 1:500 g/ha, preferably 10-70 g/ha.

The compounds of the invention are suitable for use on any soybean plant, including those that have been genetically modified to be resistant to active ingredients such as herbicides, or to produce biologically active compounds that control infestation by plant pests.

In a further preferred embodiment, transgenic plants and plant cultivars obtained by genetic engineering methods, if appropriate in combination with conventional methods (Genetically Modified Organisms), and parts thereof, are treated. Particularly preferably, plants of the plant cultivars which are in each case commercially available or in use are treated according to the invention. Plant cultivars are understood as meaning plants having novel properties (“traits”) which have been obtained by conventional breeding, by mutagenesis or by recombinant DNA techniques.

These can be cultivars, bio- or genotypes. Depending on the plant species or plant cultivars, their location and growth conditions (soils, climate, vegetation period, diet), the treatment according to the invention may also result in superadditive “synergistic”) effects.

Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products.

Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds.

Traits that are emphasized in particular are the increased defence of the plants against insects, arachnids, nematodes and slugs and snails by virtue of toxins formed in the plants, in particular those formed in the plants by the genetic material from Bacillus thuringiensis (for example by the genes Cry1A(a), Cry1A(b), Cry1A(c), Cry11A, Cry111A, CryIIIB2, Cry9c, Cry2Ab, Cry3Bb and Cry1F and also combinations thereof) (referred to herein as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins.

Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants.

Examples of “Bt plants” are soya bean varieties which are sold under the trade names YIELD GARD®

Examples of herbicide-tolerant plants which may be mentioned are soya bean varieties which are sold under the trade names Roundup Ready® (tolerance to glyphosate), Liberty Link® (tolerance to phosphinotricin), IMI® (tolerance to imidazolinones) and STS® (tolerance to sulphonylureas).

Herbicide-resistant plants (plants bred in a conventional manner for herbicide tolerance) which may be mentioned include the varieties sold under the name Clearfield® (for example maize).

Of particular interest are soybean plants carrying trains conferring resistance to 2.4D (e.g. Enlist®), glyphosate (e.g. Roundup Ready®, Roundup Ready 2 Yield®), sulfonylurea (e.g. STS®), glufosinate (e.g. Liberty Link®, Ignite®), Dicamba (Monsanto) HPPD tolerance (e.g. isoxaflutole herbicide) (Bayer CropScience, Syngenta). Double or triple stack in soybean plants of any of the traits described here are also of interest, including glyphosate and sulfonyl-urea tolerance (e.g. Optimum GAT®, plants stacked with STS® and Roundup Ready® or Roundup Ready 2 Yield®), dicamba and glyphosate tolerance (Monsanto). Soybean Cyst Nematode resistance soybean (SCN®-Syngenta) and soybean with Aphid resistant trait (AMT®—Syngneta) are also of interest.

These statements also apply to plant cultivars having these genetic traits or genetic traits still to be developed, which plant cultivars will be developed and/or marketed in the future.

The compounds of the invention may be used on soybean to control, for example, Elasmopalpus lignosellus, Diloboderus abderus, Diabrotica speciosa, Sternechus subsignatus, Formicidae, Agrotis ypsilon, Julus ssp., Anticarsia gemmatalis, Megascelis ssp., Procornitermes ssp., Gryllotalpidae, Nezara viridula, Piezodorus spp., Acrosternum spp., Neomegalotomus spp., Cerotoma trifurcata, Popillia japonica, Edessa spp., Liogenys fuscus, Euchistus heros, stalk borer, Scaptocoris castanea, phyllophaga spp., Pseudoplusia includens, Spodoptera spp., Bemisia tabaci, Agriotes spp., preferably Diloboderus abderus, Diabrotica speciosa, Nezara viridula, Piezodorus spp., Acrosternum spp., Cerotoma trifurcata, Popillia japonica, Euchistus heros, phyllophaga spp., Agriotes spp.

The compounds of the invention are preferably used on soybean to control stinkbugs, e.g. Nezara spp. (e.g. Nezara viridula, Nezara antennata, Nezara hilare), Piezodorus spp. (e.g. Piezodorus guildinii), Acrosternum spp. Euchistus spp. (e.g. Euchistus heros, Euschistus servus), Halyomorpha halys, Plautia crossota, Riptortus clavatus, Rhopalus msculatus, Antestiopsis orbitalus, Dichelops spp. (e.g. Dichelops furcatus, Dichelops melacanthus), Eurygaster spp. (e.g. Eurygaster intergriceps, Eurygaster maura), Oebalus spp. (e.g. Oebalus mexicana, Oebalus poecilus, Oebalus pugnase, Scotinophara spp. (e.g. Scotinophara lurida, Scotinophara coarctata). Preferred targets include Antestiopsis orbitalus, Dichelops furcatus, Dichelops melacanthus, Euchistus heros, Euschistus servus, Nezara viridula, Nezara hilare, Piezodorus guildinii, Halyomorpha halys. In one embodiment the stinkbug target is Nezara viridula, Piezodorus spp., Acrosternum spp, Euchistus heros. The compounds of the invention are particularly effective against Euschistus and in particular Euchistus heros. Euschistus and in particular Euchistus heros are the preferred targets.

In order to apply a compounds of the invention as an insecticide, acaricide, nematicide or molluscicide to a pest, a locus of pest, or to a plant susceptible to attack by a pest, compounds of the invention is usually formulated into a composition which includes, in addition to the compound of the invention, a suitable inert diluent or carrier and, optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of the invention. The composition is generally used for the control of pests such that a compound of the invention is applied at a rate of from 0.1 g to 10 kg per hectare, preferably from 1 g to 6 kg per hectare, more preferably from 1 g to lkg per hectare.

When used in a seed dressing, a compound of the invention is used at a rate of 0.0001 g to 10 g (for example 0.001 g or 0.05 g), preferably 0.005 g to 10 g, more preferably 0.005 g to 4 g, per kilogram of seed.

Compositions comprising a compound of the invention can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of the invention.

Dustable powders (DP) may be prepared by mixing a compound of the invention with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of the invention with one or more water-soluble inorganic salts (such as sodium bicarbonate, sodium carbonate or magnesium sulfate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of the invention with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of the invention and one or more powdered solid diluents or carriers, or from pre-formed blank granules by absorbing a compound of the invention (or a solution thereof, in a suitable agent) in a porous granular material (such as pumice, attapulgite clays, fuller's earth, kieselguhr, diatomaceous earths or ground corn cobs) or by adsorbing a compound of the invention (or a solution thereof, in a suitable agent) on to a hard core material (such as sands, silicates, mineral carbonates, sulfates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of the invention in water or an organic solvent, such as a ketone, alcohol or glycol ether. These solutions may contain a surface active agent (for example to improve water dilution or prevent crystallization in a spray tank).

Emulsifiable concentrates (EC) or oil-in-water emulsions (EW) may be prepared by dissolving a compound of the invention in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of the invention either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of the invention is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of the invention. SCs may be prepared by ball or bead milling the solid compound of the invention in a suitable medium, optionally with one or more dispersing agents, to produce a fine particle suspension of the compound. One or more wetting agents may be included in the composition and a suspending agent may be included to reduce the rate at which the particles settle. Alternatively, a compound of the invention may be dry milled and added to water, containing agents hereinbefore described, to produce the desired end product.

Aerosol formulations comprise a compound of the invention and a suitable propellant (for example n-butane). A compound of the invention may also be dissolved or dispersed in a suitable medium (for example water or a water miscible liquid, such as n-propanol) to provide compositions for use in non-pressurized, hand-actuated spray pumps.

A compound of the invention may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerization stage such that an aqueous dispersion of oil droplets is obtained, in which each oil droplet is encapsulated by a polymeric shell and contains a compound of the invention and, optionally, a carrier or diluent therefor. The polymeric shell may be produced by either an interfacial polycondensation reaction or by a coacervation procedure. The compositions may provide for controlled release of the compound of the invention and they may be used for seed treatment. A compound of the invention may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of the invention). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of the invention).

A compound of the invention may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulfuric acid (for example sodium lauryl sulfate), salts of sulfonated aromatic compounds (for example sodium dodecylbenzenesulfonate, calcium dodecylbenzenesulfonate, butylnaphthalene sulfonate and mixtures of sodium di-isopropyl- and tri-isopropyl-naphthalene sulfonates), ether sulfates, alcohol ether sulfates (for example sodium laureth-3-sulfate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulfosuccinamates, paraffin or olefine sulfonates, taurates and lignosulfonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of the invention may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapor or applied through distribution or incorporation of a composition (such as a granular composition or a composition packed in a water-soluble bag) in soil or an aqueous environment.

A compound of the invention may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs, SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of the invention (for example 0.0001 to 10%, by weight) depending upon the purpose for which they are to be used.

A compound of the invention may be used in mixtures with fertilizers (for example nitrogen-, potassium- or phosphorus-containing fertilizers). Suitable formulation types include granules of fertilizer. The mixtures preferably contain up to 25% by weight of the compound of the invention.

The invention therefore also provides a fertilizer composition comprising a fertilizer and a compound of the invention.

The compositions of this invention may contain other compounds having biological activity, for example micronutrients or compounds having fungicidal activity or which possess plant growth regulating, herbicidal, insecticidal, nematicidal or acaricidal activity.

The compound of the invention may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergize the activity or complement the activity (for example by increasing the speed of effect or overcoming repellency) of the compound of the invention; or help to overcome or prevent the development of resistance to individual components. The particular additional active ingredient will depend upon the intended utility of the composition. Examples of suitable pesticides include the following:

a) a pyrethroid including those selected from the group consisting of permethrin, cypermethrin, fenvalerate, esfenvalerate, deltamethrin, cyhalothrin, lambda-cyhalothrin, gamma-cyhalothrin, bifenthrin, fenpropathrin, cyfluthrin (including beta cyfluthrin), tefluthrin, ethofenprox, natural pyrethrin, tetramethrin, S-bioallethrin, fenfluthrin, prallethrin and 5-benzyl-3-furylmethyl-(E)-(1R,3 S)-2,2-dimethyl-3-(2-oxothiolan-3-ylidenemethyl)cyclopropane carboxylate;

b) an organophosphate including those selected from the group consisting of sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate and diazinon;

c) a carbamate including those selected from the group consisting of pirimicarb, triazamate, cloethocarb, carbofuran, furathiocarb, ethiofencarb, aldicarb, thiofurox, carbosulfan, bendiocarb, fenobucarb, propoxur, methomyl, thiodicarb and oxamyl;

d) a benzoyl urea including those selected from the group consisting of diflubenzuron, triflumuron, hexaflumuron, flufenoxuron, lufenuron and chlorfluazuron;

e) an organic tin compound selected from the group consisting of cyhexatin, fenbutatin oxide and azocyclotin;

f) a pyrazole including those selected from the group consisting of tebufenpyrad and fenpyroximate;

g) a macrolide including those selected from the group consisting of abamectin, emamectin (e.g. emamectin benzoate), ivermectin, milbemycin, spinosad, azadirachtin and spinetoram;

h) an organochlorine compound including those selected from the group consisting of endosulfan (in particular alpha-endosulfan), benzene hexachloride, DDT, chlordane and dieldrin;

i) an amidine including those selected from the group consisting of chlordimeform and amitraz;

j) a fumigant agent including those selected from the group consisting of chloropicrin, dichloropropane, methyl bromide and metam;

k) a neonicotinoid compound including those selected from the group consisting of imidacloprid, thiacloprid, acetamiprid, nitenpyram, dinotefuran, thiamethoxam, clothianidin, nithiazine and flonicamid;

l) a diacylhydrazine including those selected from the group consisting of tebufenozide, chromafenozide and methoxyfenozide;

m) a diphenyl ether including those selected from the group consisting of diofenolan and pyriproxyfen;

n) indoxacarb;

o) chlorfenapyr;

p) pymetrozine;

q) a tetramic acid compound including those selected from the group consisting of spirotetramat and spirodiclofen, or a tetronic acid compound including spiromesifen;

r) a diamide including those selected from the group consisting of flubendiamide, chlorantraniliprole (Rynaxypyr®) and cyantraniliprole;

s) sulfoxaflor;

t) metaflumizone;

u) fipronil and ethiprole;

v) pyrifluqinazon;

w) buprofezin;

x) diafenthiuron;

y) 4-[(6-Chloro-pyridin-3-ylmethyl)-(2,2-difluoro-ethyl)-amino]-5H-furan-2-one (DE 102006015467);

z) flupyradifurone.

aa) CAS: 915972-17-7 (WO 2006129714; WO2011/147953; WO2011/147952) ab) CAS: 26914-55-8 (WO 2007020986)

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethylbenzimidazole-1-sulfonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro-1-ethyl-1-methyl-2-oxopropyl)-4-methylbenzamide (RH-7281, zoxamide), N-allyl-4,5,-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5-pyridyl)-cyclopropane carboxamide, acibenzolar (CGA245704), alanycarb, aldimorph, anilazine, azaconazole, azoxystrobin, benalaxyl, benomyl, biloxazol, bitertanol, blasticidin S, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulfate, copper tallate and Bordeaux mixture, cymoxanil, cyproconazole, cyprodinil, debacarb, di-2-pyridyl disulfide 1,1′-dioxide, dichlofluanid, diclomezine, dicloran, diethofencarb, difenoconazole, difenzoquat, diflumetorim, O,O-di-iso-propyl-5-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl-(Z)-N-benzyl-N-([methyl(methyl-thioethylideneaminooxycarbonyl)amino]thio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, fluoroimide, fluquinconazole, flusilazole, flutolanil, flutriafol, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, kasugamycin, kresoxim-methyl, LY186054, LY211795, LY248908, mancozeb, maneb, mefenoxam, mepanipyrim, mepronil, metalaxyl, metconazole, metiram, metiram-zinc, metominostrobin, myclobutanil, neoasozin, nickel dimethyldithiocarbamate, nitrothal-isopropyl, nuarimol, ofurace, organomercury compounds, oxadixyl, oxasulfuron, oxolinic acid, oxpoconazole, oxycarboxin, pefurazoate, penconazole, pencycuron, phenazin oxide, phosetyl-Al, phosphorus acids, phthalide, picoxystrobin (ZA1963), poly-oxin D, polyram, probenazole, prochloraz, procymidone, propamocarb, propiconazole, propineb, propionic acid, pyrazophos, pyrifenox, pyrimethanil, pyroquilon, pyroxyfur, pyrroInitrin, quaternary ammonium compounds, quinomethionate, quinoxyfen, quintozene, sipconazole (F-155), sodium pentachlorophenate, spiroxamine, streptomycin, sulfur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb, ziram; N-[9-(dichloromethylene)-1,2,3,4-tetrahydro-1,4-methanonaphthalen-5-yl]-3-(difluoromethyl)-1-methyl-1H-pyrazole-4-carboxamide [1072957-71-1], 1-methyl-3-difluoromethyl-1H-pyrazole-4-carboxylic acid (2-dichloromethylene-3-ethyl-1-methyl-indan-4-yl)-amide, and 1-methyl-3-difluoromethyl-4H-pyrazole-4-carboxylic acid [2-(2,4-dichloro-phenyl)-2-methoxy-1-methyl-ethyl]-amide.

Preferred additional pesticidally active ingredients are those selected from neonicotinoids, pyrethroids, strobilurins, triazoles and carboxamides (SDHI inhibitors). Pyrethroids are of interest of which lambda-cyhalothrin is of particular interest. Combinations of compounds of the invention, particularly compounds from Table A, and particularly when X is P3, P4 or P5 and pyrethroids, in parrticular lambda-cyhalothrin, exhibit synergistic control of stinkbugs (according to the Colby formula), in particular Euschistus, e.g. Euschistus heros.

In a further aspect of the invention there is provided a method comprising applying to a crop of soybean plants, the locus thereof, or propagation material thereof, a combination of a compound a compound of the invention and lambda cyhalothrin in a synergistically effective amount, wherein the method is for control and/or prevention of stinkbugs, preferably Euschistus, e.g. Euschistus heros. In one embodiment the compound is a compound of formula I. In another embodiment the compound is a compound of formula II. In another embodiment the compound is a compound of formula III. In another embodiment the compound is a compound of formula IV. Preferably the compound is a compound from Table A.

The compounds of the invention may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

Unless otherwise stated the weight ratio of the compound of I, II, III or IV with an additional active ingredient may generally be between 1000:1 and 1:1000. In other embodiments that weight ratio of A to B may be between 500:1 to 1:500, for example between 100:1 to 1:100, for example between 1:50 to 50:1, for example 1:20 to 20:1, for example 1:10 to 10:1, for example 1:5 to 5:1, for example 1:1.

Compositions of the invention include those prepared by premixing prior to application, e.g. as a readymix or tankmix, or by simultaneous application or sequential application to the plant.

The invention will now be illustrated by the following non-limiting Examples. All citations are incorporated by reference.

### FIGURES

FIG. 1 shows the results of a field trial to investigate control of Euschistus heros on soybeans with the compound of formula Ix (racemic mixtures of enantiomers). The X axis indicates grams of active ingredient per hectare of (a) beta-cyfluthrin+imidacloprid, (b) thiamethoxam+lambda cyhalothrin, (c) formula Ix at 35 g/ha, (d) formula Ix at 53 g/ha. The Y axis indicates % control. Bars 1-5 show control after 1, 3, 5, 8 and 15 days after application respectively. Experimental details are indicated in Example 1.

FIG. 2 shows the results of a field trial to investigate control of Euschistus heros on soybeans with the compound of formula Ix (racemic mixtures of enantiomers). The X axis indicates grams of active ingredient per hectare of (a) beta-cyfluthrin+imidacloprid, (b) thiamethoxam+lambda cyhalothrin, (c) formula Ix at 35 g/ha, (d) formula Ix at 53 g/ha. The Y axis indicates % control. Bars 1-4 show control after 2, 5, 12 and 16 days after application respectively. Experimental details are indicated in Example 2.

FIG. 3 shows the results of a field trial to investigate control of Euschistus heros on soybeans with the compound of formula Ix (racemic mixtures of enantiomers) and formula Iy (racemix mixture of enantiomers). The X axis indicates grams of active ingredient per hectare of (a) thiamethoxam+lambda cyhalothrin, (b) methamidophos, (c) formula Iy at 60 g/ha, (d) formula Iy at 120 g/ha, (e) formula Ix at 60 g/ha, (f) formula Ix at 120 g/ha. The Y axis indicates % control. Bars 1-5 show control after 2, 5, 7, 10 and 13 days after application respectively. Experimental details are indicated in Example 3.

### EXAMPLES

### Example 1

### Field Trial

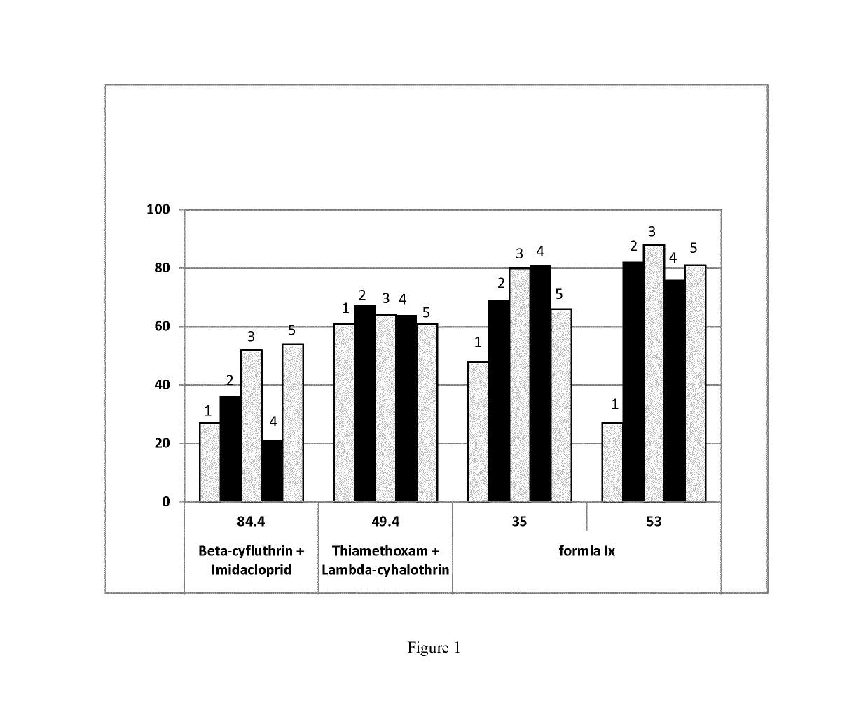

control of Euschistus heros on soybeans. Application was Foliar application using a boomsprayer. The spray volume was 200 l/ha. Each plot size was 60 m². The soybean was at growth stage bbch 72. The compound of formula Ix was applied as an EC formulation. The beta-cyfluthrin+imidacloprid (9.4 g/ha and 75 g/ha respectively)/thiamethoxam+lambda-cyhalothrin (21.2 g/ha and 28.2 g/ha respectively) were applied as suspension concentrate formulations. The results (% control of adults) are shown in FIG. 1 and represent the average of 3 replicates.

### Example 2

### Field Trial

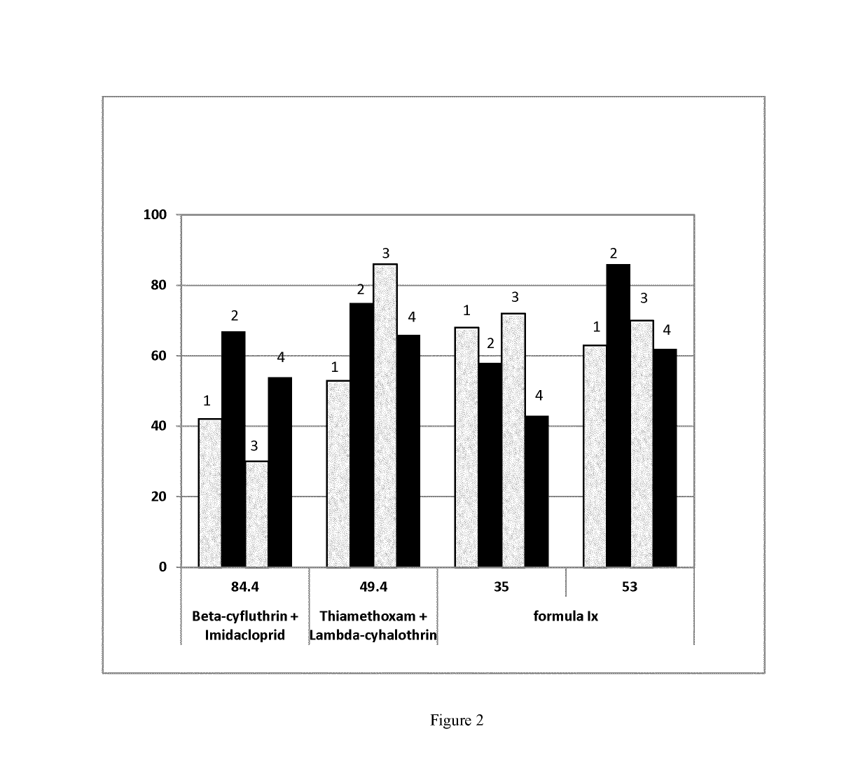

control of Euschistus heros on soybeans. Application was Foliar application using a boomsprayer. The spray volume was 200 l/ha. Each plot size was 108 m². The soybean was at growth stage bbch 72. The compound of formula Ix was applied as an EC formulation. The beta-cyfluthrin+imidacloprid (9.4 g/ha and 75 g/ha respectively)/thiamethoxam+lambda-cyhalothrin (21.2 g/ha and 28.2 g/ha respectively) were applied as suspension concentrate formulations. The results (% control of adults) are shown in FIG. 2 and represent the average of 3 replicates.

### Example 3

### Field Trial

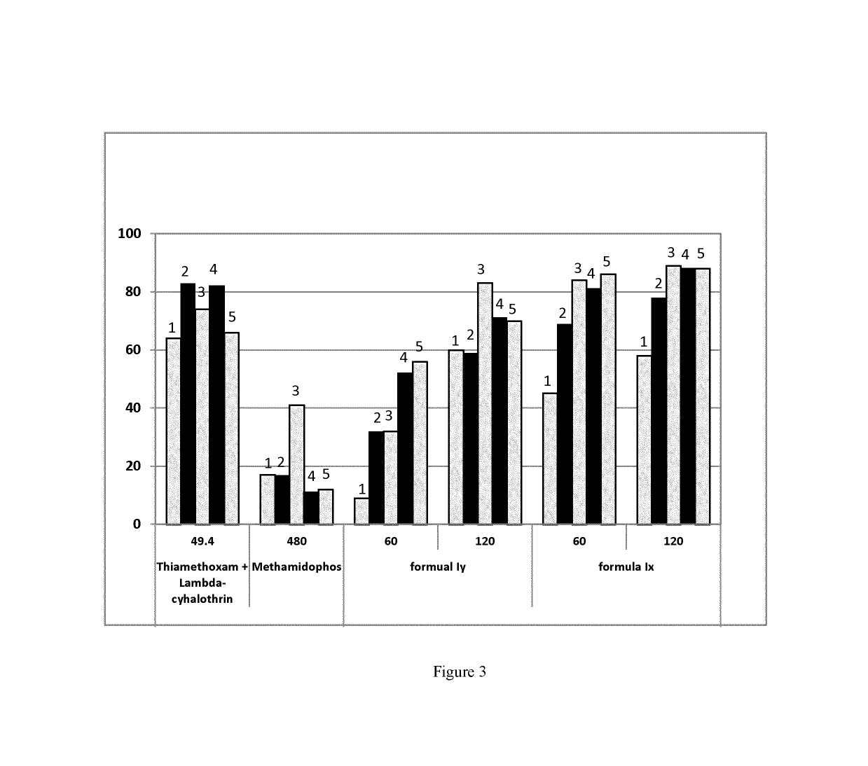

control of Euschistus heros on soybeans. Application was Foliar application using a boomsprayer. The spray volume was 200 l/ha. Each plot size was 108 m². The soybean was at growth stage bbch 75. The compounds of formula IX and Iy were applied as an EC formulation. The thiamethoxam+lambda-cyhalothrin (21.2 g/ha and 28.2 g/ha respectively) was applied as a suspension concentrate formulation. The methamidophos was applied as a soluble concentrate formulation. The results (% control of adults) are shown in FIG. 3 and represent the average of 3 replicates.

### Example 4

### Euschistus heros (Neotropical Brown Stink Bug) (Contact/Feeding Activity)

2 week old soybean plants are sprayed in a turn table spray chamber with the diluted spray solutions. After drying, 2 soybean seeds are added and plants are infested with 10 N-2 nymphs of the neotropical brown stink bug Euschistus heros in plastic test boxes. Boxes are incubated in a climate chamber at 25° C. and 60% RH. Evaluation is done 5 days after infestation on mortality and growth effects. The results are shown in Tables E1 and E2 below. The data is an average of two replicates.

The results show that the compounds of the invention are significantly more active against stinkbugs than structurally similar compounds, particularly at low rates of application.

TABLE E1

| | | | Compound 3 | Compound 4 |
|---|---|---|---|---|
| | | | (comparative | (comparative |
| Rate/ppm | Compound 1 | Compound 2 | example) | example) |
| 12.5 | 85 | 95 | 75 | 35 |
| 6 | 80 | 95 | 20 | 25 |
| 3 | 65 | 65 | 0 | 15 |

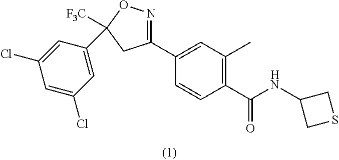 [SMILES: Cc1cc(C2=NOC(c3cc(Cl)cc(Cl)c3)(C(F)(F)F)C2)ccc1C(=O)NC1CSC1]

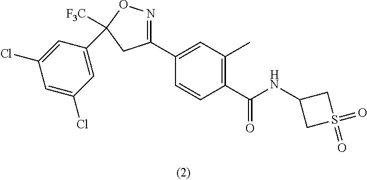 [SMILES: Cc1cc(C2=NOC(c3cc(Cl)cc(Cl)c3)(C(F)(F)F)C2)ccc1C(=O)NC1CS(=O)(=O)C1]

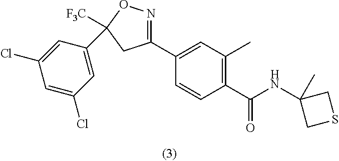 [SMILES: Cc1cc(C2=NOC(c3cc(Cl)cc(Cl)c3)(C(F)(F)F)C2)ccc1C(=O)NC1(C)CSC1]

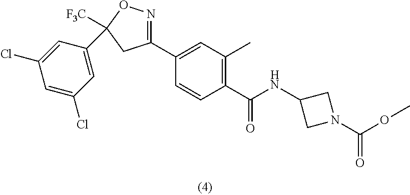 [SMILES: COC(=O)N1CC(NC(=O)c2ccc(C3=NOC(c4cc(Cl)cc(Cl)c4)(C(F)(F)F)C3)cc2C)C1]

Compounds 1-4 are disclosed in WO2009/080250.

TABLE E2

| | | | Compound 7 | Compound 8 | Compound 9 |
|---|---|---|---|---|---|
| Rate/ | Com- | Com- | (comparative | (comparative | (comparative |
| ppm | pound 5 | pound 6 | example) | example) | example) |
| 12.5 | 100 | 95 | 60 | 95 | 70 |
| 6 | 90 | 100 | 20 | 35 | 75 |
| 3 | 85 | 95 | 5 | 5 | 15 |

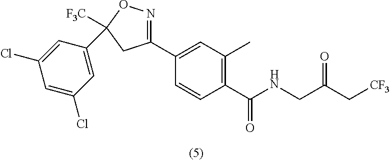 [SMILES: Cc1cc(C2=NOC(c3cc(Cl)cc(Cl)c3)(C(F)(F)F)C2)ccc1C(=O)NCC(=O)CC(F)(F)F]

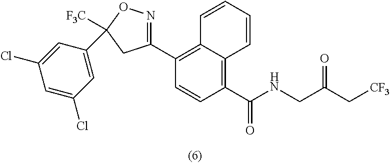 [SMILES: O=C(CNC(=O)c1ccc(C2=NOC(c3cc(Cl)cc(Cl)c3)(C(F)(F)F)C2)c2ccccc12)CC(F)(F)F]

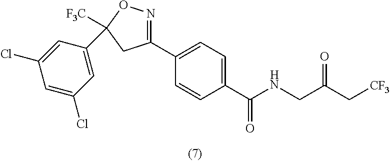 [SMILES: O=C(CNC(=O)c1ccc(C2=NOC(c3cc(Cl)cc(Cl)c3)(C(F)(F)F)C2)cc1)CC(F)(F)F]

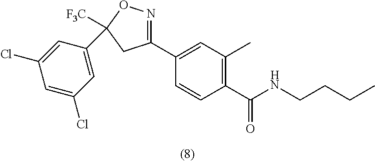 [SMILES: CCCCNC(=O)c1ccc(C2=NOC(c3cc(Cl)cc(Cl)c3)(C(F)(F)F)C2)cc1C]

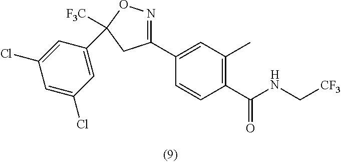 [SMILES: Cc1cc(C2=NOC(c3cc(Cl)cc(Cl)c3)(C(F)(F)F)C2)ccc1C(=O)NCC(F)(F)F]

Compounds 5-9 are compounds from WO2005/085216 (EP1731512) and WO2009/002809.

### REFERENCES

Correa-Perreira, B. S.; Panizzi, A. R., Percevejos da soja e seu manejo, Londrina: Embrapa-CNPSo, 1999, 45 (Circular Técnica, 24).
Galileo, M. H. M., Heinrichs E. A., Retenção foliar em plantas de soja (glycine max (1.) merrill) resultantes da ação de Piezodorus guildinii (Westwood, 1837) (Hemiptera pentatomidae), em diferentes niveis e épocas de infestação. An. Soc. Entomol. Brasil, 1978, 7, 85-98.
Panizzi, A. R., Slansky junior, F. Review of phytophagous pentatomids (Hemiptera pentatomidae) associated with soybean in the Americas, Florida Entomologist, Gainesville, 1985, 68(1), 184-214.
Schmidt, F. G. V., Pires, C. S. S., Sujii, E. R., Borges, M., Pantaleão, D. C., Lacerda, A. L., Azevedo, C. R., Comportamento e captura das fêmeas de Euschistus heros em armadilhas iscadas com feromonio sexual., 2003, Comunicado Técnico 93. Brasilia, DF.
Sosa-Gomez, D. R., Silva, J. Da., Lopes, I. O, N., Corso, I., Almeida, A. M. R. Almeida, moraes, g. c.p.m.; baur, m. insecticide susceptibility of Euschistus heros (Heteroptera pentatomidae) in Brazil, Journal of Economic Entomology, 2009, 102(3), 1209-1216.
Todd, J. W., Herzog, D. C., Sampling phytophagous pentatomidae on soybean. in: Kogan, M., Herzog, D. C. (ed.). Sampling methods in soybean entomology, New York: Springer, 1980, 438-478.

1. A method comprising applying to a crop of soybean plants, the locus thereof, or propagation material thereof, a compound of formula I 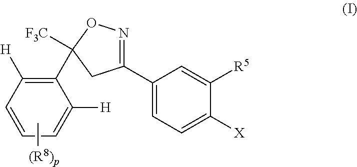
wherein X is P1 or P2 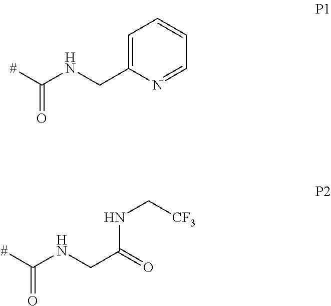
R⁵ is chloro, bromo, CF₃ or methyl;
each R⁸ is independently bromo, chloro, fluoro or trifluoromethyl;
p is 1, 2 or 3;
and wherein the method is for controlling and/or preventing infestation of the soybean crop by Euschistus.

2. (canceled)

3. A method according to claim 1, wherein the compound of formula I is a compound of formula Ix

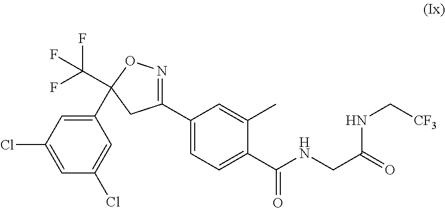 [SMILES: Cc1cc(C2=NOC(c3cc(Cl)cc(Cl)c3)(C(F)(F)F)C2)ccc1C(=O)NCC(=O)NCC(F)(F)F]

4. A method comprising applying to a crop of soybean plants, the locus thereof, or propagation material thereof, a compound of formula II 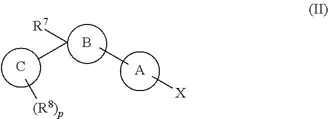
wherein
cycle A is A2a 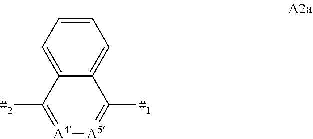
wherein A^{4′} and A^{5′} are independently C—H, or nitrogen and wherein #1 indicates the bond to X and #2 indicates the bond to cycle B;
cycle B is selected from B1 to B6 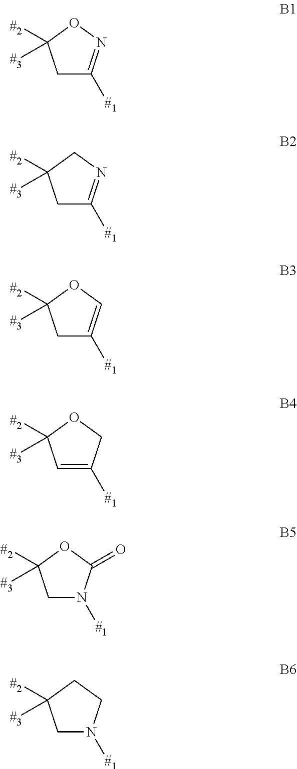
wherein #1 indicates the bond to cycle A, #2 indicates the bond to R⁷ and #3 indicates the bond to cycle C;
cycle C is phenyl;
R⁷ is chlorodifluoromethyl or trifluoromethyl;
each R⁸ is independently bromo, chloro, fluoro or trifluoromethyl;
p is 2 or 3; and
X is P1 or P2 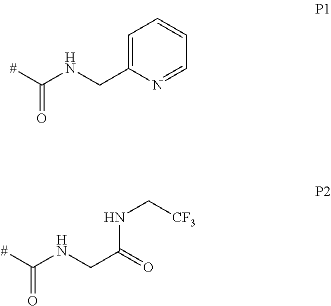
and wherein the method is for controlling and/or preventing infestation of the soybean crop by stinkbugs.

5. A method comprising applying to a crop of soybean plants, the locus thereof, or propagation material thereof, a compound of formula III 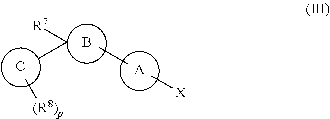
wherein
cycle A is A1 a or A2a 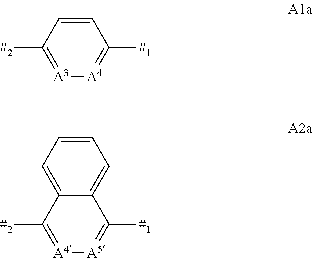
wherein A³, A⁴, A^{4′} and A^{5′} are independently C—H, or nitrogen and wherein #1 indicates the bond to X and #2 indicates the bond to cycle B;
cycle B is selected from B1 to B6 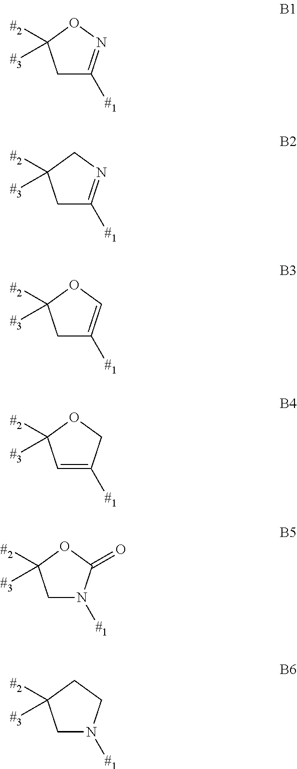
wherein #1 indicates the bond to cycle A, #2 indicates the bond to R⁷ and #3 indicates the bond to cycle C;
cycle C is phenyl;
R⁵ is chloro, bromo, CF₃ or methyl;
R⁷ is chlorodifluoromethyl or trifluoromethyl;
each R⁸ is independently bromo, chloro, fluoro or trifluoromethyl;
p is 2 or 3;
and X is selected from P3 to P11

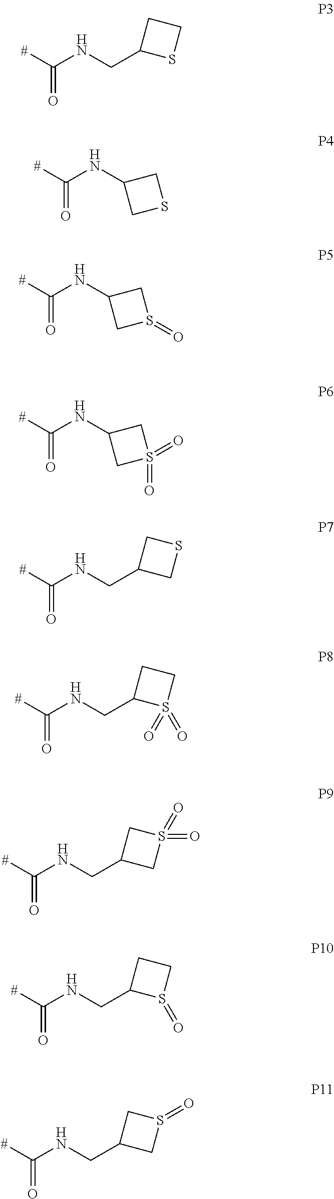

6. A method according to claim 5, wherein the method is for controlling and/or preventing infestation of the soybean crop by stinkbugs.

7. (canceled)

8. A method according to claim 5, wherein X is P4, P5 or P6.

9. A method according to claim 4, wherein cycle B is cycle B1.

10. A method according to claim 5, wherein the compound of formula III is a compound of formula A 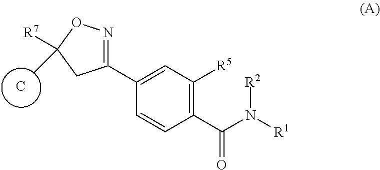
wherein G is oxygen, R⁷ is trifluoromethyl, R⁵ is methyl, R¹ is hydrogen, and cycle C and R² have the values:

| | Cycle C | R² |
|---|---|---|
| 1 | 3,5-dichloro-phenyl- | thietan-3-yl- |
| 2 | 3,5-dichloro-phenyl- | 1-oxo-thietan-3-yl- |
| 3 | 3,5-dichloro-phenyl- | 1,1-dioxo-thietan-3-yl- |
| 4 | 3,5-dichloro-phenyl- | thietan-2-yl-methyl- |
| 5 | 3,5-dichloro-phenyl- | 1-oxo-thietan-2-yl-methyl- |
| 6 | 3,5-dichloro-phenyl- | 1,1-dioxo-thietan-2-yl-methyl- |
| 7 | 3,5-dichloro-phenyl- | thietan-3-yl-methyl- |
| 8 | 3,5-dichloro-phenyl- | 1-oxo-thietan-3-yl-methyl- |
| 9 | 3,5-dichloro-phenyl- | 1,1-dioxo-thietan-3-yl-methyl- |

11. A method according to claim 4, wherein stinkbug is Nezara viridula, Piezodorus spp., Acrosternum spp, Euchistus heros.

12. A method according to claim 4, wherein stinkbug is Euschistus.

13. A method according to claim 1, wherein Euschistus is Euschistus heros.

14. A method or use according to claim 1, wherein the compound of formula I or formula II is applied in combination with one or more additional active ingredients selected from neonicotinoids, pyrethroids, strobilurins, triazoles and carboxamides.

15. A method according to claim 1, wherein the compound is applied to the crop by foliar application.

16. A method according to claim 1, wherein the compound of formula I or formula II is applied in combination with an attractant selected from glucose, saccharose, salt, glutamate, citric acid, soybean oil, peanut oil and soybean milk.
